SEQUENCE LISTING (56 sequences)

SEQ ID NO: 1 - protein, 14 aa, synthetic construct
SVTEQGAELSNEER

SEQ ID NO: 2-33 - skipped (32 entries)

SEQ ID NO: 34 - protein, 4 aa, synthetic construct
SVTE

SEQ ID NO: 35 - protein, 5 aa, synthetic construct
SVTEQ

SEQ ID NO: 36 - protein, 6 aa, synthetic construct
SVTEQG

SEQ ID NO: 37 - protein, 7 aa, synthetic construct
SVTEQGA

SEQ ID NO: 38 - protein, 4 aa, synthetic construct
EQGA

SEQ ID NO: 39 - protein, 6 aa, synthetic construct
VTEQGA

SEQ ID NO: 40 - protein, 6 aa, synthetic construct
SVTQAA

SEQ ID NO: 41 - protein, 14 aa, synthetic construct
TAVLENSREGESQE

SEQ ID NO: 42-46 - skipped (5 entries)

SEQ ID NO: 47 - protein, 7 aa, synthetic construct
ELSNEER

SEQ ID NO: 48 - protein, 6 aa, synthetic construct
CSVTCG

SEQ ID NO: 49 - protein, 7 aa, synthetic construct
  VARIANT 1: note = S may be replaced by T
  VARIANT 2: note = V may be replaced by L, F, W or Y
  VARIANT 3: note = T may be replaced by S
  VARIANT 5: note = Q may be replaced by N
  VARIANT 7: note = A may be replaced by V, L or F
SVTXQGA

SEQ ID NO: 50 - protein, 7 aa, synthetic construct
  VARIANT 2: note = S may be replaced by T
  VARIANT 3: note = V may be replaced by L, F, W or Y
  VARIANT 4: note = T may be replaced by S
  VARIANT 5: note = Q may be replaced by N
  VARIANT 7: note = A may be replaced by V, L or F
XSVTQGA

SEQ ID NO: 51 - protein, 7 aa, synthetic construct
  VARIANT 1: note = S may be replaced by T
  VARIANT 2: note = V may be replaced by L, F, W or Y
  VARIANT 3: note = T may be replaced by S
  VARIANT 4: note = Q may be replaced by N
  VARIANT 6: note = A may be replaced by V, L or F
SVTQGAX

SEQ ID NO: 52 - protein, 7 aa, synthetic construct
  VARIANT 1: note = Q may be replaced by N
  VARIANT 3: note = A may be replaced by V, L or F
  VARIANT 6: note = V may be replaced by L, F, W or Y
  VARIANT 7: note = T may be replaced by S
  VARIANT 5: note = S may be replaced by T
QGAXSVT

SEQ ID NO: 53 - protein, 7 aa, synthetic construct
  VARIANT 4: note = A may be replaced by V, L or F
  VARIANT 5: note = S may be replaced by T
  VARIANT 6: note = V may be replaced by L, F, W or Y
  VARIANT 7: note = T may be replaced by S
  VARIANT 2: note = Q may be replaced by N
XQGASVT

SEQ ID NO: 54 - protein, 7 aa, synthetic construct
  VARIANT 1: note = Q may be replaced by N
  VARIANT 3: note = A may be replaced by V, L or F
  VARIANT 4: note = S may be replaced by T
  VARIANT 5: note = V may be replaced by L, F, W or Y
  VARIANT 6: note = T may be replaced by S
QGASVTX

SEQ ID NO: 55 - protein, 6 aa, synthetic construct
  VARIANT 1: note = S may be replaced by T
  VARIANT 2: note = V may be replaced by L, F, W or Y
  VARIANT 3: note = T may be replaced by S
  VARIANT 4: note = Q may be replaced by N
  VARIANT 6: note = A may be replaced by V, L or F
SVTQGA

SEQ ID NO: 56 - protein, 6 aa, synthetic construct
  VARIANT 1: note = Q may be replaced by N
  VARIANT 3: note = A may be replaced by V, L or F
  VARIANT 4: note = S may be replaced by T
  VARIANT 5: note = V may be replaced by L, F, W or Y
  VARIANT 6: note = T may be replaced by S
QGASVT

(19) United States
(12) Patent Application Publication
(10) Pub. No.: US 2025/0361271 A1
(43) Pub. Date: Nov. 27, 2025

(54) PEPTIDE AGONIST

(71) Applicant: THE UNIVERSITY OF BIRMINGHAM, Birmingham (GB)

(72) Inventors: Asif IQBAL, Birmingham (GB); Helen MCGETTRICK, Birmingham (GB); George Edward RAINGER, Birmingham (GB)

(73) Assignee: THE UNIVERSITY OF BIRMINGHAM, Birmingham (GB)

(21) Appl. No.: 19/099,324

(22) PCT Filed: Jul. 28, 2023
(86) PCT No.: PCT/GB2023/052013

§ 371 Date: Jan. 28, 2025

(30) Foreign Application Priority Data
Jul. 28, 2022 (GB) 2211043.1

(51) Int. Cl. C07K 7/06; A61K 38/00; A61P 19/02; A61P 29/00

(52) U.S. Cl. CPC C07K 7/06; A61P 19/02; A61P 29/00; A61K 38/00

(57) ABSTRACT

The present teaching relates to peptides that are capable of regulating the trafficking of leukocytes. This has applications in the treatment and/or prophylaxis of conditions associated with leukocyte migration, including inflammatory diseases and/or musculoskeletal (MSK) loss and/or damage.

### FIELD

The present teaching relates to peptides that are capable of regulating the trafficking of leukocytes. This has applications in the treatment and/or prophylaxis of conditions associated with leukocyte migration, including inflammatory diseases and/or musculoskeletal (MSK) loss and/or damage.

### BACKGROUND

Targeting inflammation in chronic disease represents a huge unmet need in clinical practice. This has been recognized for many years, however efforts to develop anti-inflammatory drugs have largely targeted molecules that promote inflammation. It is now known that many of these agents are functionally redundant, and 30 years of effort has yielded very few efficacious drugs. Moreover, even when inflammation is well controlled, bone damage can continue, and reparative normal bone formation remains poor. Thus, new strategies are required to develop effective treatments of inflammation and/or of MSK loss and/or damage.

Exaggerated or prolonged inflammation and leukocyte trafficking and retention in affected tissues are the hallmark of many common disorders, including inflammation such as immune mediated inflammatory diseases (IMIDs), that can lead to disability and death. This means that restitution of control over the inflammatory response represents a realistic target for therapeutic intervention in such diseases. Despite this, the in-built regulators of immune cell trafficking remain poorly understood.

Studies carried out by Chimen et al in Nat. Med., 2015, 21 (5), 467-475, have identified a peptide released following the stimulation of B cells with adiponectin. This peptide is capable of regulating T-cell trafficking in inflammatory conditions and is known as the peptide inhibitor of trans-endothelial migration, or PEPITEM (SEQ ID NO. 1).

PEPITEM comprises 14-amino acids and is a proteolytically derived peptide that spans residues 28 to 41 of its parent protein, 14-3-3 zeta/delta (14-3-3ζδ), which comprises 245 amino acids and is a product of the YWHAZ gene. 14-3-3 proteins contain seven isoforms and are known to be expressed in all eukaryotic cells. The family is capable of binding to a multitude of signalling molecules, such as kinases, phosphatases and transmembrane receptors, highlighting their ability to influence and regulate processes such as the cell cycle (see Bridges, D. and Moorhead, G. B., Sci. STKE, 2005, 296, re10; and Wilker, E. and Yaffe, M. B., J. Mol. Cell Cardiol., 2004, 37, 633-42).

PEPITEM was first described as being secreted from B cells and inhibiting T-cell transmigration. Studies carried out using an in vitro model of transmigration (Chimen et al, supra.) suggest that the secretion of PEPITEM from B-cells following their treatment with adiponectin could regulate the transmigration of peripheral blood lymphocytes (PBLs) through human umbilical vein endothelial cells (HUVECs) stimulated with TNF-α and IFN-γ. Chimen et al. report that PEPITEM has efficacy in murine models of Sjogren's disease, uveitis (ocular disease), septicaemia and ischaemia/reperfusion injury. Using a biotin-conjugate on the N-terminal of PEPITEM, Chimen et al. identified cadherin-15 (CDH-15) as the endothelial receptor of PEPITEM. CDH-15 is a transmembrane glycoprotein which functions as a Ca²⁺ dependent cell adhesion molecule.

In WO 2013/104928 (The University of Birmingham), it is described that PEPITEM supports a homeostatic pathway which works to limit the magnitude of the inflammatory response irrespective of how it is initiated. The efficacy of PEPITEM across such a broad spectrum of IMIDs makes a strong case for the translation potential of the pathway.

In WO 2018/165218 (Allysta Pharmaceuticals, Inc.), PEPITEM is reported to be useful in the treatment of dry eye and ocular diseases of inflammation.

Abnormalities in the bones or joints of individuals underpin pathology in musculoskeletal (MSK) diseases, such as inflammatory arthritidies, osteoporosis, cancer-induced bone disease, Paget's disease of bone and the rare groups of metabolic bone diseases; where patients suffer permanent loss of function and pain. Moreover, patients with rheumatoid arthritis, psoriatic arthritis and/or osteoarthritis are likely to suffer from inflammation induced bone damage resulting in the need for joint replacement surgery. Sedentary activity where the bones are not actively loaded, such as prolonged bed-rest (>5 days) due to, for example, disease/surgery and hospitalisation or space travel, leads to loss in bone mass.

Maintenance of bone integrity is a key medical challenge, especially in ageing populations. MSK diseases affect >10 million people in the UK, costing the NHS ˜£4.7 billion per year and accounting for over 30 million working days lost per annum (Musculoskeletal data Advisory group response to the Government's mandate to NHS England 2017/18). Existing therapies focus on reducing joint pain and/or slowing the rate of bone damage, whilst therapies inducing bone repair and limiting bone loss are often ignored.

Bone growth and repair is dependent predominantly on the activities of osteoblast and osteoclast cells (see Raggatt, L. J., J. Biol. Chem., 2010, 285, 25103-25108). Osteoblast cells are the major cellular component of bone and almost the entire bone matrix in a mammal is mineralised by osteoblasts. Osteoblasts synthesise and mineralise bone during both bone formation and bone remodelling. In contrast, osteoclasts break down and restructure bone tissue by producing enzymes that dissolve the collagen, calcium and phosphorus of the bone. Currently, anti-resorptive bisphosphonates are typically used to treat osteoporosis, which inhibit bone resorption by promoting apoptosis of osteoclasts. However, long-term use is associated with increased incidence of micro-fractures and atypical femur fractures, suggesting that these drugs may hinder normal bone remodelling and repair (see, for example, Haworth, A. E. and Webb, J. Br. J. Radiol., 2002, 85 (1018), 1333-1342). Newer drugs on the market include anti-RANKL antibody (denosumab); an src kinase inhibitor (saracatinib); and a cathespin K inhibitor (odanacatib), which was discontinued in 2016 due to increased risk of stroke (see Hanley, D. A. et al., Int. J. Clin. Pract., 2012, 66 (12), 1139-1146; Danson, S. et al., J. Bone Oncol., 2019, 19, 100261; Bromme, D. and Lecaille, F., Expert Opin. Investig. Drugs, 2009, 18 (5), 585-600). These agents help to reduce the rate of bone damage by altering the activity of osteoclasts and preventing bone resorption. However, none affect osteoblasts—the cells known to induce bone formation.

Methods of reducing bone loss and/or stimulating bone production by controlling the balance between osteoclast and osteoblast activity are likely to be useful in the treatment of MSK diseases and/or damage, including any disorder of accelerated bone loss or impaired bone remodelling, such as cancer-induced bone disease, Paget's disease of bone and the rare groups of metabolic bone diseases, or diseases associated with inflammation (e.g RA, OA). Agents that stimulate bone formation, i.e. which stimulate osteoblast activity, are likely to be particularly effective in such treatment, since bone formation and mineralisation would not be limited by the natural, potentially under-active activity of osteoblasts.

In U.S. 62/912,439 (The University of Birmingham), it is described that PEPITEM is effective in reducing bone loss and/or stimulating bone production when administered to a patient and/or bone cells in effective amounts.

In view of the above, PEPITEM has the potential to treat both inflammation and MSK damage simultaneously, providing an advantage over other therapies where either inflammation or MSK damage is treated by a drug, thus patients require two different drugs, one per condition.

Although PEPITEM has been identified as efficacious against T-cell trafficking via interactions with CDH-15 and in reducing bone loss and/or stimulating bone growth, the core functional motif present in the peptide that mediates its function has not been reported. A fuller understanding of the core functional motif of PEPITEM is required to take full advantage of its potential as a regulator of leukocyte trafficking and an inhibitor of bone loss/promotor of bone growth, and to take advantage of it being easier and cheaper to manufacture than peptides of longer lengths (such as PEPITEM). The present invention aims to address this.

### SUMMARY

The inventors have found that peptides described herein are surprisingly effective regulators of leukocyte trafficking and stimulators of osteoblast activity. The peptides comprise no fewer than 3 and no more than 7 amino acids. Consequently, they are relatively short peptides and may be advantageously easier and cheaper to manufacture than peptides of longer lengths, such as PEPITEM. Some of the peptides of the disclosure have surprising additional advantages over PEPITEM as a regulator of leukocyte trafficking, such as increased efficacy, i.e. smaller half maximal inhibitory concentration (IC₅₀) values, longer half-lives (T_{1/2}) in plasma, better amenability to transport by peptide transporters, better solubility and/or, in some instances, better stability.

The skilled person is aware that any reference to an aspect of the current disclosure may include any embodiment of that aspect. For example, any reference to the first aspect may include the first aspect and any embodiments of the first aspect.

Viewed from a first aspect, there is provided a peptide of 3-7 amino acid residues for use as a medicament wherein the peptide comprises formula (I) and/or formula (II); wherein formula (I) is:

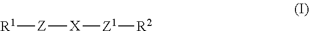

wherein Z and Z¹ are each independently an amino acid selected from serine and threonine; X is an amino acid selected from valine, leucine, phenylalanine, tryptophan and tyrosine; R¹ is hydrogen (H), COR³ or a bond to another amino acid; R² is OH (hydroxy), N(R⁴)₂ or a bond to another amino acid; and each R³ and R⁴ is independently selected from hydrogen (H) and a C₁-C₆ alkyl group; and
formula (II) is:

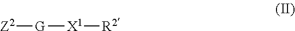

wherein Z² is selected from N, Q, Ac—N, Ac-Q (CH₃CO-Q) or pyroglutamic acid (pE), wherein Q represents glutamine and N represents asparagine; G represents glycine; X¹ is an amino acid selected from alanine, valine, leucine, phenylalanine and 2-amino-2-methylpropanoic acid; R^{2′} is OH (hydroxy), N(R^{4′})₂ or a bond to another amino acid; and each R⁴ is independently selected from hydrogen (H) and a C₁-C₆ alkyl group; and
wherein the peptide is not of the sequence CSVTCG, i.e. Cys-Ser-Val-Thr-Cys-Gly (SEQ ID NO. 48).

Viewed from a second aspect, there is provided the peptide of the first aspect for use in regulating leukocyte migration.

Viewed from a third aspect, there is provided the peptide of the first aspect for use in the treatment and/or prophylaxis of inflammation and/or musculoskeletal (MSK) loss and/or damage.

Viewed from a fourth aspect, there is provided a method of reducing bone loss and/or stimulating bone production, the method comprising administering an effective amount of the peptide of the first aspect ex vivo directly to bone cells and/or their precursors.

Viewed from a fifth aspect, there is provided a peptide of 3-7 amino acid residues as defined in the first aspect, wherein the peptide is not of sequence SVT, i.e. Ser-Val-Thr (SEQ ID NO. 2).

Viewed from a sixth aspect, there is provided a pharmaceutical composition comprising a therapeutically effective amount of the peptide of the first or fourth aspect and a pharmaceutically acceptable excipient.

### BRIEF DESCRIPTION OF THE FIGURES

FIG. 1 is a schematic of the mechanism by which PEPITEM regulates the trafficking of T cells.

FIG. 2 contains two histograms of the adhesion of PBL and the transmigration of PBL across cytokine-stimulated HDBECs on addition of PEPITEM, its derivatives and peptides disclosed herein. **P≤0.01, ***P≤0.001 and Ns=not statistically significant by one-way ANOVA followed by a Dunnett's post-test compared to untreated controls (PBL0 and scrambled peptide). Data are represented as mean±S.E.M of ≥3 experiments.

FIGS. 3 (A) and (B) are graphs comparing (A) the total adherence and (B) transmigration of PBLs across HDBECs on addition of SVT and QGA at concentrations increasing from 0.0001 to 500 ng/ml. Ns=not statistically significant by one-way ANOVA followed by a Dunnett's post-test when compared to an untreated control (PBL0). Data are represented as mean±S.E.M of ≥3 experiments

FIG. 4 contains two histograms of the adhesion of PBL and the transmigration of PBL across cytokine-stimulated HDBECs on addition of peptides in which at least one amino acid of the SVT and QGA sequences is substituted with alanine. **P≤0.01 and ns=not statistically significant when analysed by one-way ANOVA with Dunnett's post-test compared to an untreated control (PBL0). Data are represented as mean±S.E.M of 3 experiments.

FIG. 5a contains two histograms comparing the alkaline phosphatase (ALP) activity in primary murine calvarial osteoblasts (left histogram) and primary human osteoblasts (right histogram) that are untreated, or treated separately with SVT, QGA, SVTEQGA or ELSNEER for 4 days in the presence of osteoblast differentiation media. ALP activity directly correlates with the amount of bone mineral produced by osteoblasts (i.e. their activity). A greater ALP activity indicates greater osteoblast activity, more mineral and thus more bone formed. Data are represented as mean±S.E.M of 6 (murine osteoblasts) or 4 (human osteoblasts) experiments.

FIG. 5b contains two histograms comparing the alkaline phosphatase (ALP) activity in primary murine calvarial osteoblasts that are untreated, or treated separately with PEPITEM, SVT, SVT-NH-Ethyl, TSV or QGA for 8 days in the presence of osteoblast differentiation media.

FIG. 6 is a histogram comparing the knee thickness of mice injected with antigen (BSA) into a maximum of two sites in the lower dorsal area and are then treated separately with PBS (control), SVT, QGA, or PEPITEM for 4 days. Data are represented as mean±S.E.M of n experiments.

FIG. 7 contains four histograms comparing the number of leukocytes counted in the peritoneum of mice after 4 hours of treatment with Zymosan and either no peptide (control) or one of SVT-[NH-Ethyl], [pGlu]-GA-amide and TVS-amide. Top left histogram shows the numbers of CD45⁺ cells, top right histogram shows the number of CD11c⁺ cells, bottom left histogram shows the number of F4/80^{hi} cells and bottom right histogram shows the number of SiglecF⁺ cells. A lower cell number corresponds to reduced trafficking of leukocytes and a reduced inflammatory response.

FIG. 8 contains a histogram (top) comparing the number of Ly6G⁺ cells counted in the peritoneum of mice after 4 hours of treatment with Zymosan and either no peptide (control) or one of SVT-[NH-Ethyl], [pGlu]-GA-amide and TVS-amide. A lower cell number corresponds to reduced trafficking of Ly6G⁺ cells and a reduced inflammatory response. FIG. 8 also contains two flow cytometry scatter graphs (bottom) the number of LY6G⁺ cells, where the left-hand graph shows the response on treatment with Zymosan and PBS (control) and the right-hand graph shows the response on treatment with Zymosan and SVT.

FIG. 9 contains line graphs (a) to (d) comparing joint inflammation (a) and (c) and ankle swelling (b) and (d) arising from injection of monosodium urate (MSU) in mice when treated with PEPITEM, SVT-[NH-Ethyl] or Ac-QGA-Acid.

FIG. 10 contains histograms comparing the dose dependent effects of PEPITEM, SVT and QGA on the recruitment of (a) T cells (CD3⁺), (b) B cells (CD19⁺) and (c) neutrophils to the murine peritoneum during sterile peritonitis after administration of zymosan.

FIG. 11 contains a histogram comparing the severity of imiquimod-induced plaque psoriasis in mice after seven days during which some of the mice received topical treatment with PEPITEM, Ac-QGA-Acid or SVT-[NH-Ethyl].

FIG. 12 contains histograms comparing IL-6 and TNF-α concentrations in the supernatants of murine macrophage cells 24 hours after the addition of LPS (a phlogistic stimulus) to the cells, where some of the cells had been pretreated with a control peptide, PEPITEM or peptides disclosed herein.

FIG. 13 contains histograms comparing IL-6 and TNF-α concentrations in the supernatants of murine macrophage cells 24 hours after the addition of LPS (a phlogistic stimulant) to the cells, where some of the cells had been pretreated with a control peptide, PEPITEM or peptides disclosed herein.

FIG. 14 contains a histogram comparing cell proliferation of keratinocytes 72 hours after the addition of M5 cytokines (a proliferation stimulant) to the cells, where some of the cells had been pretreated with a control peptide, PEPITEM or peptides disclosed herein.

FIG. 15 contains a histogram comparing neutrophil adhesion to TNF-α-stimulated endothelial cells 15 minutes after the addition of neutrophils to the TNF-α-stimulated endothelial cells, where either the neutrophils or the endothelial cells had been pretreated with PEPITEM or peptides disclosed herein, or had not been pretreated.

### DETAILED DESCRIPTION

The peptides of the disclosure comprise no fewer than 3 and no more than 7 amino acids and in certain embodiments are surprisingly effective regulators of leukocyte trafficking and inhibitors of bone loss/promotors of bone growth. They may be advantageously easier and cheaper to manufacture than peptides of longer lengths, such as PEPITEM, and some may exhibit increased efficacy, i.e. smaller IC₅₀ values, longer T_{1/2} values in plasma, better amenability to transport by peptide transporters, better solubility and/or in some cases better stability than PEPITEM.

In the discussion that follows, reference is made to a number of terms, which have the meanings provided below, unless a context indicates to the contrary. The nomenclature used herein for defining compounds, in particular the compounds disclosed herein, is generally based on the rules of the IUPAC organisation for chemical compounds, specifically the “IUPAC Compendium of Chemical Terminology (Gold Book)”. For the avoidance of doubt, if a rule of the IUPAC organisation is contrary to a definition provided herein, the definition herein is to prevail.

The term “comprising” or variants thereof is understood to imply the inclusion of a stated element, integer or step, or group of elements, integers or steps, but not the exclusion of any other element, integer or step, or group of elements, integers or steps.

The term “consisting” or variants thereof is understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within ±5% of the value specified. For example, if the level of inhibition of leukocyte migration is such that migration is reduced by at least about 30%, a reduction of 31.5% and a reduction of 28.5% is included.

The term “alkyl” is well known in the art and defines univalent groups derived from alkanes by removal of a hydrogen atom from any carbon atom, wherein the term “alkane” is intended to define cyclic or acyclic branched or unbranched hydrocarbons having the general formula CₙH₂ₙ₊₂, wherein n is an integer ≥1. A C₁₋₆alkyl group is an alkyl group having from one to six carbon atoms. Examples of alkyl groups include methyl, ethyl, propyl, isopropyl, butyl, isobutyl, sec-butyl, tert-butyl, pentyl, isopentyl, tert-pentyl, hexyl, and isohexyl.

As is known in the art, 2-amino-2-methylpropanoic acid (also known as α-aminoisobutyric acid (AIB), α-methylalanine or 2-methylalanine) is a non-proteinogenic amino acid with the structural formula NH₂C(CH₃)₂COOH.

As is known in the art, pyroglutamic acid (pE) is a natural amino acid derivative of glutamine or glutamic acid, wherein the N-terminal amino group attacks the carbonyl group of the side chain in a nucleophilic substitution reaction, in which the side-chain amino group of glutamine or side-chain hydroxy group of glutamic acid is displaced, and a lactam forms.

The term “enantiomer” defines one of a pair of molecular entities that are mirror images of each other and non-superimposable, i.e. cannot be brought into coincidence by translation and rigid rotation transformations. Enantiomers are chiral molecules, i.e. are distinguishable from their mirror image.

The term “half maximal inhibitory concentration value” or “IC₅₀ value” is used herein to refer to the amount of peptide required to inhibit T-cell migration by 50% of the maximum inhibition observed.

The term “half-life” or “T_{1/2}” is used to define the time taken for the concentration of the peptide in the blood or plasma to be reduced by 50%.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “treatment” defines the therapeutic treatment of a human or non-human animal, in order to impede or reduce or halt the rate of the progress of the condition, or to ameliorate or cure the condition. Prophylaxis of the condition as a result of treatment is also included. References to prophylaxis are intended herein not to require complete prevention of a condition: its development may instead be hindered through treatment in accordance with the invention. Typically, treatment is not prophylactic, and the compound or composition is administered to a patient having a diagnosed or suspected condition. By an “effective amount” herein defines an amount of the peptide or composition disclosed herein that is sufficient to impede the noted diseases and thus produces the desired therapeutic or inhibitory effect. The skilled person is aware that the effective amount of peptide is not restricted to amounts that lead to overall improvement of a condition. Rather, the effective amount includes amounts that reduce the rate of deterioration of a condition. The skilled person is further aware that an effective amount is likely to vary with the particular compound disclosed herein, the subject and the administration procedure used. It is within the means and capacity of the skilled person to identify the effective amount of the compounds and compositions via routine work and experimentation. Typically, the effective amount may lie within a range of 1 mg/Kg to 100 mg/Kg.

The term “prodrug” is used herein to refer to a compound which acts as a drug precursor and which, upon administration to a subject, undergoes conversion by metabolic or other chemical processes to yield a peptide disclosed herein.

The term “pharmaceutically acceptable excipient” defines substances other than a pharmacologically active drug or prodrug, which are included in a pharmaceutical product.

The term “intrathecal administration” defines administration of a compound by injection into the spinal canal, or into the subarachnoid space.

The term “intraosseous administration” defines administration of a compound by injection into the bone marrow.

The term “intravenous administration” defines administration of a compound by injection into a vein or veins.

The term “intramuscular administration” defines administration of a compound by injection into a muscle.

The term “subcutaneous administration” defines administration of a compound by injection into the subcutis, i.e. the layer of skin directly below the dermis and epidermis.

The term “oral administration” defines administration of a compound through the mouth, wherein the compound is typically in the form of a tablet or capsule.

“Prolonged bed rest” is used herein to refer to bed rest for a period of time ranging from several days to several months. The skilled person is aware that a patient is not necessarily immobile for the entirety of the period, or confined to bed because of a health impairment that physically prevents them from leaving bed. However, the patient is necessarily in bed for the majority of the period.

The term “biocompatible” is used herein to refer to a material that is not harmful or toxic to living tissue.

The peptide chains of all formulae herein, such as formulae (I) and (II) (Z—X—Z¹ and Z²-G-X¹), are written from the N-terminus to the C-terminus.

The standard single letter and three letter codes for amino acids are used herein and are as follows: A (Ala) Alanine; C (Cys) Cysteine; D (Asp) Aspartic acid; E (Glu) Glutamic acid; F (Phe) Phenylalanine; G (Gly) Glycine; H (His) Histidine; I (Ile) Isoleucine; K (Lys) Lysine; L (Leu) Leucine; M (Met) Methionine; N (Asn) Asparagine; P (Pro) Proline; Q (Gln) Glutamine; R (Arg) Arginine; S (Ser) Serine; T (Thr) Threonine; V (Val) Valine; W (Trp) Tryptophan; Y (Tyr) Tyrosine. It is to be understood that reference herein to serine, threonine and/or tyrosine includes both the unmodified amino acid and the amino acid modified such that the proton on the hydroxyl group of the side chain is replaced with a C₁₋₄alkyl group, such as methyl or ethyl. In some embodiments, reference to serine, threonine and/or tyrosine refers to both the unmodified amino acid and the amino acid modified such that the proton on the hydroxyl group of the side chain is replaced with a methyl. In other embodiments, reference to serine, threonine and/or tyrosine refers to the unmodified amino acid.

All amino acids but glycine comprise a chiral centre at the α-carbon atom, i.e. the carbon atom positioned between the C- and N-termini. The amino acids found in eukaryotic cells are L-amino acids, i.e. levo-amino acids, which are able to rotate polarized light in an anti-clockwise direction. D-amino acids, i.e. dextro-amino acids, are able to rotate polarized light in a clockwise direction and are found in some prokaryotic cells. The amino acids of the peptide may be in the D or L configuration. The amino acids of the peptide may all be in the same configuration, or some amino acids may have a different configuration to the others. For example, one amino acid may be in the L-form, while the other amino acids are in the D-form. In some embodiments, all of the amino acids in the peptide are in the L-form. In some embodiments, all of the amino acids in the peptide are in the D-form.

It is to be understood that amino acids situated within the peptide are bonded to each other by peptide bonds, which form via condensation reactions between the C-terminal carboxylic acid group of one amino acid and the N-terminal amino group of another amino acid. Thus, where a feature positioned within a peptide is referred to herein as an amino acid, it is to be understood that said feature is an amino acid residue, wherein the OH of the C-terminal carboxylic acid is replaced with a bond to the N-terminal nitrogen atom of another amino acid, and a proton on the N-terminal amino acid is replaced with a bond to the C-terminal carbon atom of another amino acid. Where an amino acid is positioned at the beginning of a peptide, it is to be understood that the OH of the C-terminal carboxylic acid is replaced with a bond to the N-terminal nitrogen atom of another amino acid, and that a proton on the N-terminal amino acid may be replaced with a different moiety, such as an acetyl group. Furthermore, where an amino acid is positioned at the end of a peptide, it is to be understood that one of the protons on the N-terminal amino acid is replaced with a bond to the C-terminal carbon atom of another amino acid, and that the OH of the C-terminal carboxylic acid may be replaced with a different moiety, such as an amino group.

For the avoidance of doubt, the definitions of R¹, R², R^{2′}, R³, R⁴ and R^{4′} are chemical formulae, i.e. elements are referred to rather than amino acids.

The first aspect provides a peptide of 3-7 amino acid residues (a 3mer, 4mer, 5mer, 6mer or 7mer) for use as a medicament, wherein the peptide comprises formula (I) and/or formula (II), and wherein the peptide is not of the sequence CSVTCG.

Formula (I) is:

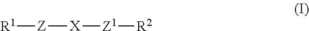

wherein Z and Z¹ are each independently an amino acid selected from serine and threonine; X is an amino acid selected from valine, leucine, phenylalanine, tryptophan and tyrosine; R¹ is hydrogen (H), COR³ or a bond to another amino acid; R² is OH, N(R⁴)₂ or a bond to another amino acid; and each R³ and R⁴ is independently selected from H and a C₁-C₆ alkyl group.
Formula (II) is:

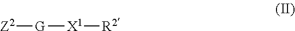

wherein Z² is selected from N, Q, Ac—N, Ac-Q (H₃C—CO-Q) or pyroglutamic acid, wherein Q represents glutamine and N represents asparagine; G represents glycine; X¹ is an amino acid selected from alanine, valine, leucine, phenylalanine and 2-amino-2-methylpropanoic acid; R^{2′} is OH, N(R^{4′})₂ or a bond to another amino acid; and each R⁴ is independently selected from H and a C₁-C₆ alkyl group.

In formula (I), the R¹ group is bound to the alpha nitrogen of the N-terminal amino acid (Z) of the peptide. Thus, in embodiments wherein R¹ is hydrogen, it will be understood that the N-terminal amine group is unmodified (i.e. it is a primary amine).

In some embodiments, the peptide is a 6mer or 7mer comprising a peptide of formula (I), together with a peptide of formula (II). In such embodiments, formula (I) and formula (II) may be present in any order: formula (I) may be positioned first followed by formula (II) or vice versa. Where formula (I) is positioned first (before formula (II)), R² is a bond to another amino acid and Z² is selected from N or Q. Where formula (II) is positioned first (before formula (I)), R^{2′} and R¹ are each a bond to another amino acid and Z² is selected from Ac—N, Ac-Q or pyroglutamic acid.

In some embodiments, when the peptide is a 7mer, it is represented by any one of formulae (IIIa) to (IIIf):

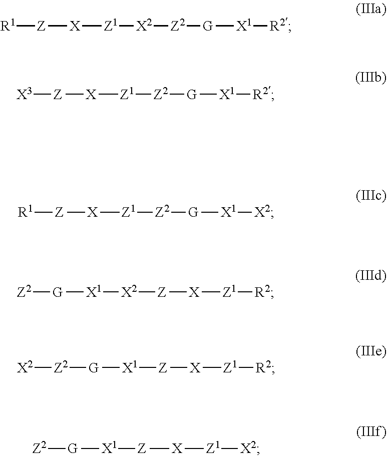

wherein X² and X³ are each an amino acid. Formulae (IIIa) to (IIIc) represent a 7mer in which formula (I) appears first, R² of formula (I) is a bond between Z¹ and X² or Z¹ and Z², represented by —, and Z² is N or Q. In formula (IIIa), R¹ is H or COR³, and R^{2′} is OH or N(R^{4′})₂. In formula (IIIb), R¹ of formula (I) is a bond between X³ and Z, represented by —, and R^{2′} is OH or N(R^{4′})₂. In formula (IIIc), R^{2′} of formula (II) is a bond between X¹ and X², represented by — and R¹ is H or COR³.

Formulae (IIId) to (IIIf) represent a 7mer in which formula (II) appears first, and R^{2′} of formula (II) is a bond between X¹ and X² or X¹ and Z, represented by —. R¹ is a bond between X² and Z or X¹ and Z, represented by —. In formula (IIId), R² is OH or N(R⁴)₂ and Z² is selected from Ac—N, Ac-Q or pyroglutamic acid. In formula (IIIe), X¹ is bonded directly to Z, Z² is N or Q, and R² is OH or N(R⁴)₂. In formula (IIIf), X¹ is bonded directly to Z, R² of formula (II) is a bond between Z¹ and X², represented by —, and Z² is selected from Ac—N, Ac-Q or pyroglutamic acid.

In some embodiments, when the peptide is a 7mer it is represented by any one of formulae (IIIa) to (IIIc), often (IIIa).

When the peptide is a 6mer comprising a peptide of formula (I), together with a peptide of formula (II), it is represented by formula (IVa) or formula (IVb):

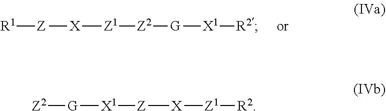

Formula (IVa) represents a 6mer in which formula (I) appears first, R² of formula (I) is a bond between Z¹ and Z², represented by —, R¹ is H or COR³, Z² is N or Q, and R^{2′} is OH or N(R^{4′})₂. Formula (IVb) represents a 6mer in which formula (II) appears first, R^{2′} of formula (II) is a bond between X¹ and Z, represented by —, Z² is selected from Ac—N, Ac-Q or pyroglutamic acid, and R² is OH or N(R⁴)₂.

In some embodiments, where the peptide is a 6mer comprising a peptide of formula (I) together with a peptide of formula (II), it is represented by formula (IVa).

In particular embodiments, when the peptide is a 6mer it comprises a peptide of formula (I) together with a peptide of formula (II).

In some embodiments, the peptide comprises formula (I) or formula (II). In such embodiments, where the peptide comprises 4 amino acids, formula (I) or formula (II) may be at the beginning or at the end of the peptide. Where the peptide comprises more than 4 amino acids, formula (I) or formula (II) may be at the beginning, within, or at the end of the peptide.

Where formula (I) is at the beginning of the peptide, R² is a bond to another amino acid and R¹ is H or COR³. Where formula (I) is within the peptide, R¹ and R² are each a bond to another amino acid. Where formula (I) is at the end of the peptide, R¹ is a bond to another amino acid and R² is OH or N(R⁴)₂.

Where formula (II) is at the beginning of the peptide, R^{2′} is a bond to another amino acid, and Z² is selected from Ac—N, Ac-Q or pyroglutamic acid. Where formula (II) is within the peptide, Z² is N or Q and R^{2′} is a bond to another amino acid. Where formula (II) is at the end of the peptide, Z is N or Q and R^{2′} is OH or N(R^{4′})₂.

When the peptide is a 3mer comprising formula (I) or formula (II), it is represented by formula (I) or formula (II), wherein R¹ is hydrogen (H) or COR³; R² is OH or N(R⁴)₂; Z² is selected from Ac—N, Ac-Q or pyroglutamic acid, and R^{2′} is OH or N(R^{4′})₂.

In some embodiments, when the peptide is a 4mer comprising formula (I) or formula (II), it is represented by any one of formulae (Va) to (Vd):

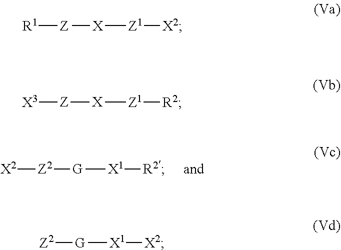

wherein R¹ is hydrogen (H) or COR³; X² and X³ are each an amino acid; R² is OH or N(R⁴)₂; Z² of formula (Vc) is N or Q; R^{2′} is OH or N(R^{4′})₂ and Z² of formula (Vd) is selected from Ac—N, Ac-Q or pyroglutamic acid.

In some embodiments, when the peptide is a 4mer, it is represented by any one of formulae (Va), (Vc) and (Vd).

In some embodiments, when the peptide is a 5mer comprising formula (I) or formula (II), it is represented by any one of formulae (VIa) to (VIf):

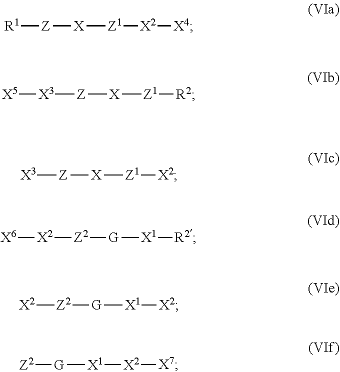

wherein R¹ is hydrogen (H) or COR³; X² to X⁷ are each an amino acid; R² is OH or N(R⁴)₂; Z² of formulae (VId) and (VIe) is N or Q; R^{2′} is OH or N(R^{4′})₂ and Z² of formula (VIf) is selected from Ac—N, Ac-Q or pyroglutamic acid.

In some embodiments, when the peptide is a 5mer comprising formula (I) or formula (II), X³ is an amino acid other than cysteine, X² is an amino acid other than cysteine and/or X⁴ is an amino acid other than glycine. In some cases, when the peptide a 5mer comprising formula (I) or formula (II), X² is glutamic acid and/or X⁴ is glutamine.

In some embodiments, when the peptide is a 5mer, it is represented by any one of formulae (VIa) to (VIf), provided it is not of the sequence TTSYT.

In particular embodiments, when the peptide is of formula (VIb), X is not tyrosine, i.e. X is an amino acid selected from valine, leucine, phenylalanine and tryptophan.

In even more particular embodiments, when the peptide is a 5mer, X is not tyrosine, i.e. X is an amino acid selected from valine, leucine, phenylalanine and tryptophan.

In some embodiments, when the peptide is a 5mer, it is represented by any one of formulae (VIa), and (VIc) to (VIf).

In some embodiments, when the peptide is a 5mer, it is represented by any one of formulae (VIa), and (VId) to (VIf).

In some embodiments, when the peptide is a 6mer comprising formula (I) or formula (II), it is represented by any one of formulae (VIIa) to (VIIh):

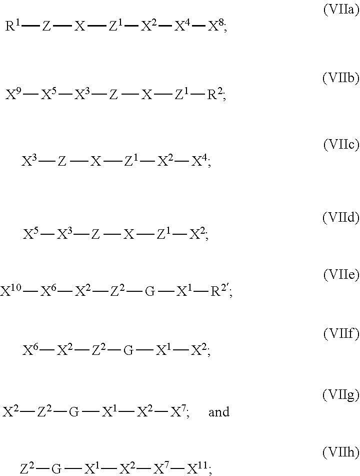

wherein R¹ is hydrogen (H) or COR³; X² to X¹¹ are each an amino acid; R² is OH or N(R⁴)₂; Z² of formulae (VIIe) to (VIIg) is N or Q; R^{2′} is OH or N(R⁴)₂ and Z² of formula (VIIh) is selected from Ac—N, Ac-Q or pyroglutamic acid.

For the avoidance of doubt, when the peptide is a 6mer comprising formula (I) or formula (II), it is not of the sequence CSVTCG. In some cases, when the peptide is represented by formula (VIIc), X³ is an amino acid other than cysteine, X² is an amino acid other than cysteine and/or X⁴ is an amino acid other than glycine. In some cases, when the peptide is represented by formula (VIIc), X² is glutamic acid and/or X⁴ is glutamine.

In some embodiments, when the peptide is a 6mer comprising formula (I) or formula (II), X³ is an amino acid other than cysteine, X² is an amino acid other than cysteine and/or X⁴ is an amino acid other than glycine. In some cases, when the peptide a 6mer comprising formula (I) or formula (II), X² is glutamic acid and/or X⁴ is glutamine.

In some embodiments, when the peptide is a 6mer, it is represented by any one of formulae (VIIa), and (VIIe) to (VIIh).

In particular embodiments, when the peptide is a 6mer, it is represented by any one of formulae (VIIc) and (VIIe) to (VIIh).

In some embodiments, when the peptide is a 7mer comprising formula (I) or formula (II), it is represented by any one of formulae (VIIIa) to (VIIIh):

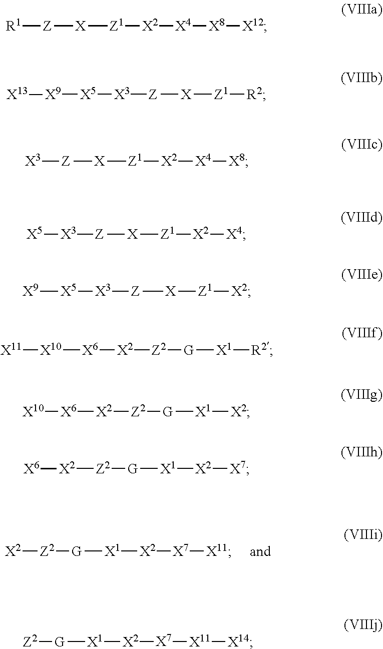

wherein R¹ is hydrogen (H) or COR³; X² to X¹⁴ are each an amino acid; R² is OH or N(R⁴)₂; Z² of formulae (VIIIf) to (VIIIi) is N or Q; R^{2′} is OH or N(R^{4′})₂ and Z² of formula (VIIIj) is selected from Ac—N, Ac-Q or pyroglutamic acid.

In some embodiments, when the peptide is a 7mer comprising formula (I) or formula (II), it does not comprise the sequence CSVTCG. In some cases, when the peptide is represented by formula (VIIIc) or (VIIId), X³ is an amino acid other than cysteine, X² is an amino acid other than cysteine and/or X⁴ is an amino acid other than glycine. In some cases, when the peptide is represented by formula (VIIIc) or (VIIId), X² is glutamic acid and/or X⁴ is glutamine.

In some embodiments, when the peptide is a 7mer comprising formula (I) or formula (II), X³ is an amino acid other than cysteine, X² is an amino acid other than cysteine and/or X⁴ is an amino acid other than glycine. In some cases, when the peptide a 7mer comprising formula (I) or formula (II), X² is glutamic acid and/or X⁴ is glutamine.

In some embodiments, when the peptide is a 7mer, it is represented by any one of formulae (VIIIa), and (VIIIf) to (VIIIj).

In some embodiments, X² is any one selected from the group consisting of glutamic acid and aspartic acid. Typically, X² is glutamic acid.

In some embodiments, X⁴ is any one selected from the group consisting of glutamine and asparagine. Typically, X⁴ is glutamine.

In some embodiments, X³ is an amino acid other than cysteine, X² is an amino acid other than cysteine and/or X⁴ is an amino acid other than glycine. In some cases, X² is glutamic acid and/or X⁴ is glutamine.

In some embodiments, X⁶ is any one selected from the group consisting of threonine and serine. Typically, X⁶ is threonine.

In some embodiments, X⁷ is any one selected from the group consisting of leucine, isoleucine, valine, alanine, methionine, phenylalanine, tyrosine and tryptophan, such as leucine, isoleucine, valine or alanine. Typically, X⁷ is leucine.

In some embodiments, X⁸ is glycine.

In some embodiments, X¹⁰ is any one selected from the group consisting of valine, leucine, phenylalanine, tryptophan and tyrosine, such as valine, leucine or phenylalanine.

Typically, X¹⁰ is valine.

In some embodiments, X¹¹ is any one selected from the group consisting of serine or threonine. Typically, X¹¹ is serine.

In some embodiments, X¹² is any one selected from the group consisting of alanine, valine, leucine, phenylalanine and 2-amino-2-methylpropanoic acid, such as alanine, valine or leucine. Typically, X¹² is alanine.

In some embodiments, X¹⁴ is any one selected from the group consisting of asparagine and glutamine. Typically, X¹⁴ is asparagine.

As defined above, formula (I) is represented by:

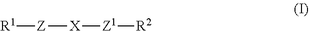

wherein Z and Z¹ are each independently an amino acid selected from serine and threonine; X is an amino acid selected from valine, leucine, phenylalanine, tryptophan and tyrosine; R¹ is H, COR³ or a bond to another amino acid; R² is OH, N(R⁴)₂ or a bond to another amino acid; and each R³ and R⁴ is independently selected from H and a C₁-C₆ alkyl group. In some embodiments, Z is serine. In some embodiments, X is an amino acid selected from valine, leucine, phenylalanine and tryptophan. In other embodiments, X is selected from valine, tryptophan and tyrosine. In some embodiments, X is selected from valine and tryptophan.

In some embodiments, Z¹ is threonine. In other embodiments, Z¹ is serine.

In some embodiments, Z is threonine, X is valine and Z¹ is serine.

As described herein, the amino acids of the peptide may be in the D or L configuration. In some embodiments, Z is threonine, X is valine and Z¹ is serine, wherein each amino acid is in the D configuration.

In some embodiments, R³ is a C₁₋₆alkyl group. Often, R³ is a C₁₋₄alkyl group, i.e. any one selected from the group consisting of methyl, ethyl, n-propyl, isopropyl, n-butyl, sec-butyl, isobutyl and tert-butyl. Typically, R³ is methyl (CH₃ or ‘Me’). In such embodiments, R¹ may be selected from H, COCH₃ and a bond to another amino acid. It will be appreciated that when R³ is methyl, R¹ is an acetyl (Ac) group (—COCH₃).

In some embodiments, R¹ is selected from H and COR³, such as H or COCH₃. In such embodiments, formula (I) is positioned at the beginning of the peptide, i.e. Z is the first amino acid of the peptide.

Each R⁴ is independently selected from H and an alkyl group having from one to six carbon atoms. Thus, R² may be NH₂, NHR⁴ or N(R⁴)₂. In some embodiments, each R⁴ is independently selected from H and a C₁₋₄alkyl group, i.e. any one selected from any one of the group consisting of H, methyl, ethyl, n-propyl, isopropyl, n-butyl, sec-butyl, isobutyl and tert-butyl. Often, each R⁴ is independently selected from any one of the group consisting of H, methyl and ethyl. In some embodiments, at least one R⁴ is H, i.e. N(R⁴)₂ is NH₂ or NH (R⁴) for example, N(R⁴)₂ is NH₂, NH(CH₃) or NH(CH₂CH₃). In some embodiments, R⁴ is ethyl, for example, R² is NH (ethyl). In some embodiments, R⁴ is hexyl, for example, R² is NH (hexyl). Typically, N(R⁴)₂ is NH₂ or NH(CH₂CH₃).

The R² group is bound to the carbon of the alpha carboxyl group of the C-terminal amino acid (Z¹). Thus, in embodiments wherein R² is OH, a carboxylic acid is provided at the C-terminus of the peptide and in embodiments wherein R² is N(R⁴)₂, an amide is provided at the C-terminus of the peptide. In some embodiments, R² is selected from OH, NH₂, NH(CH₃), NH(CH₂CH₃) and a bond to another amino acid. In other embodiments, R² is selected from OH, NH₂, NH(CH₂CH₃) and a bond to another amino acid. Where formula (I) is positioned at the end of the peptide, i.e. where Z¹ is the final amino acid of the peptide, R² is not a bond to another amino acid. In such cases, R² is typically selected from OH, NH₂ and NH(CH₂CH₃), such as NH₂ and NH(CH₂CH₃).

In some embodiments, R¹ is H or COCH₃ and R² is NH₂ or NHR⁴, e.g. NH (ethyl).

In some embodiments, Z—X—Z¹ is selected from SVT; SLT; SFT; SWT; SYT; TVT; SVS; and TVS. Sometimes, Z—X—Z¹ is selected from SVT; SWT; SYT; TVT; SVS; and TVS.

As defined above, formula (II) is represented by:

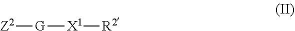

wherein Z² is selected from N, Q, Ac—N, Ac-Q or pyroglutamic acid, wherein Q represents glutamine and N represents asparagine; G represents glycine; X¹ is an amino acid selected from alanine, valine, leucine, phenylalanine and 2-amino-2-methylpropanoic acid; R^{2′} is OH, N(R^{4′})₂ or a bond to another amino acid; and each R^{4′} is independently selected from H and a C₁-C₆ alkyl group.

Where formula (II) is positioned at the beginning of the peptide, i.e. where Z² is the first amino acid of the peptide, Z² is any one selected from the group consisting of Ac—N, Ac-Q or pyroglutamic acid. Where formula (II) is not positioned at the beginning of the peptide, i.e. where Z² is not the first amino acid of the peptide, Z² is selected from N or Q.

In some embodiments, X¹ is an amino acid selected from alanine, valine, leucine and phenylalanine. In other embodiments, X¹ is an amino acid selected from alanine and leucine. Typically, X¹ is alanine. In some embodiments, X¹ is leucine.

Each R^{4′} is independently selected from H and an alkyl group having from one to six carbon atoms. Thus, R^{2′} may be NH₂, NHR^{4′} or N(R⁴)₂. In some embodiments, each R^{4′} is independently selected from H and a C₁₋₄alkyl group, i.e. any one selected from any one of the group consisting of H, methyl, ethyl, n-propyl, isopropyl, n-butyl, sec-butyl, isobutyl and tert-butyl. Often, each R^{4′} is independently selected from any one of the group consisting of H, methyl and ethyl. In some embodiments, at least one R^{4′} is H, i.e. N(R^{4′})₂ is NH₂ or NH (R⁴), for example N(R⁴)₂ is NH₂, NH(CH₃) or NH(CH₂CH₃). In some embodiments, R^{4′} is methyl, for example, R^{2′} is NH(CH₃) or N(CH₃)₂. In other embodiments, R^{4′} is ethyl, for example, R^{2′} is NH(CH₂CH₃). Often, N(R^{4′})₂ is NH₂, N(CH₃)₂, NH(CH₃) or NH(CH₂CH₃). Sometimes, N(R⁴)₂ is NH₂, N(CH₃)₂ or NH(CH₃). Typically, N(R^{4′})₂ is NH₂ or N(CH₃)₂.

The R^{2′} group is bound to the carbon of the alpha carboxyl group of the C-terminal amino acid (X¹). Thus, in embodiments wherein R^{2′} is OH, a carboxylic acid is provided at the C-terminus of the peptide and in embodiments wherein R^{2′} is N(R^{4′})₂, an amide is provided at the C-terminus of the peptide. In some embodiments, R^{2′} is selected from OH, NH₂, N(CH₃)₂, NH(CH₃), NH(CH₂CH₃) and a bond to another amino acid. In other embodiments, R^{2′} is selected from NH₂, N(CH₃)₂, NH(CH₃), NH(CH₂CH₃) and a bond to another amino acid, for example R^{2′} is selected from NH₂, N(CH₃)₂ and a bond to another amino acid. Where formula (II) is positioned at the end of the peptide, i.e. where X¹ is the final amino acid of the peptide, R^{2′} is not a bond to another amino acid. In such cases, R^{2′} is typically selected from NH₂, and N(CH₃)₂.

In some embodiments, Z² is any one selected from the group consisting of Ac—N, Ac-Q or pyroglutamic acid and R^{2′} is selected from NH₂, and N(CH₃)₂.

In some embodiments, Z²-G-X¹ is selected from QGA; NGA; Ac-QGA; pEGA; Ac-QGAib; Ac-NGA; pEGV; pEGL; and pEGF. In some embodiments, Z²-G-X¹ is selected from Ac-QGA; PEGA; Ac-NGA; pEGV; pEGL; and pEGF.

In some embodiments, the peptide comprises three to five amino acids. For example, the peptide may be a 3mer or 4mer.

In some embodiments, the peptide is a 3mer represented by formula (I), wherein R¹ is hydrogen (H) or COR³ and R² is OH or N(R⁴)₂.

In some embodiments, the tripeptide is any one selected from those listed in Table 1:

TABLE 1

| Ref. | Structure | MW | R¹ | Z | X | Z¹ | R² |
|---|---|---|---|---|---|---|---|
| SEQ ID NO. 2 | H-SVT | 305.33 | H | Ser | Val | Thr | OH |
| SEQ ID NO. 3 | Ac-SVT | 347.37 | Ac | Ser | Val | Thr | OH |
| SEQ ID NO. 4 | H-SVT-NH₂ | 304.35 | H | Ser | Val | Thr | NH₂ |
| SEQ ID NO. 5 | Ac-SVT-NH₂ | 346.38 | Ac | Ser | Val | Thr | NH₂ |
| SEQ ID NO. 6 | H-SVT-NH-Ethyl | 332.40 | H | Ser | Val | Thr | NH-ethyl |
| SEQ ID NO. 7 | H-S*VT-NH₂ | 304.35 | H | Ser | Val | Thr | NH₂ |
| SEQ ID NO. 8 | H-TVT-NH₂ | 318.37 | H | Thr | Val | Thr | NH₂ |
| SEQ ID NO. 9 | H-SVS-NH₂ | 290.32 | H | Ser | Val | Ser | NH₂ |
| SEQ ID NO. 10 | H-S*V*T*-NH₂ | 304.35 | H | Ser | Val | Thr | NH₂ |
| SEQ ID NO. 11 | H-T*V*S*-NH₂ | 304.35 | H | Thr | Val | Ser | NH₂ |
| SEQ ID NO. 12 | H-S(OMe)VT-NH₂ | 318.37 | H | Ser^{#} | Val | Thr | NH₂ |
| SEQ ID NO. 13 | H-SVT-NH-Hexyl | 388.51 | H | Ser | Val | Thr | NH-hexyl |
| SEQ ID NO. 14 | H-SIT-NH₂ | 318.37 | H | Ser | Ile | Thr | NH₂ |
| SEQ ID NO. 15 | H-SLT-NH₂ | 318.37 | H | Ser | Leu | Thr | NH₂ |
| SEQ ID NO. 16 | H-SFT-NH₂ | 352.39 | H | Ser | Phe | Thr | NH₂ |
| SEQ ID NO. 17 | H-SWT-NH₂ | 391.43 | H | Ser | Trp | Thr | NH₂ |
| SEQ ID NO. 18 | H-SYT-NH₂ | 368.39 | H | Ser | Tyr | Thr | NH₂ |

*D-isomers
^{#}“OMe” indicates that the OH side of the serine residue has been methylated

In some embodiments, the tripeptide is selected from the group consisting of SEQ ID NO. 2 to 6, 8, 9, 11, and 15 to 18.

In some embodiments, the tripeptide is selected from the group consisting of SEQ ID NO. 4, 5, 9, 11 and 17.

In some embodiments, the peptide is a 3mer represented by formula (II), wherein Z² is selected from Ac—N, Ac-Q or pyroglutamic acid and R^{2′} is OH or N(R^{4′})₂.

In some embodiments, the tripeptide is any one selected from those listed in Table 2:

TABLE 2

| Ref. | Structure | MW | Z² | X¹ | R^{2′} |
|---|---|---|---|---|---|
| SEQ ID NO. 19 | Ac-QGA | 316.31 | Acetyl-Q | Ala | OH |
| SEQ ID NO. 20 | Ac-QGA-NH₂ | 315.33 | Acetyl-Q | Ala | NH₂ |
| SEQ ID NO. 21 | pEGA | 257.25 | PyroE | Ala | OH |
| SEQ ID NO. 22 | pEGA-NH-Ethyl | 284.32 | PyroE | Ala | NH(ethyl) |
| SEQ ID NO. 23 | pEGA-NH₂ | 256.26 | PyroE | Ala | NH₂ |
| SEQ ID NO. 24 | Ac-QGA-NH-Ethyl | 343.38 | Acetyl-Q | Ala | NH(ethyl) |
| SEQ ID NO. 25 | Ac-QGAib | 330.34 | Acetyl-Q | 2-amino-2-methyl- | OH |
| | | | | propanoic acid | |
| SEQ ID NO. 26 | Ac-Q*GA*-NH₂ | 315.33 | Acetyl-Q | Ala* | NH₂ |
| SEQ ID NO. 27 | Ac-NGA | 301.30 | Acetyl-N | Ala | OH |
| SEQ ID NO. 28 | pE*GA*-NH₂ | 256.26 | PyroE | Ala* | NH₂ |
| SEQ ID NO. 29 | pEGA-NHMe | 270.29 | PyroE | Ala | NHMe |
| SEQ ID NO. 30 | pEGA-N(Me)₂ | 284.32 | PyroE | Ala | N(Me)₂ |
| SEQ ID NO. 31 | pEGV-NH₂ | 284.32 | PyroE | Val | NH₂ |
| SEQ ID NO. 32 | pEGL-NH₂ | 298.34 | PyroE | Leu | NH₂ |
| SEQ ID NO. 33 | pEGF-NH₂ | 332.36 | PyroE | Phe | NH₂ |

*D-isomers

In some embodiments, the tripeptide is selected from the group consisting of SEQ ID NO. 19 to 23, 25, 26, and 28 to 33.

In some embodiments, the tripeptide is selected from the group consisting of SEQ ID NO. 23, 26, 28, 31 and 32.

In some embodiments, the peptide is of 4-7 amino acid residues and comprises formula (I) and/or formula (II).

In some embodiments, the peptide is any one selected from those listed in Table 3:

TABLE 3

| Ref. | Structure | MW | R¹ | Z | X | Z¹ | X² | X⁴ | X⁶ | X⁸ | X¹⁰ | Z² | X¹ | R² |
|---|---|---|---|---|---|---|---|---|---|---|---|---|---|---|
| SEQ ID NO. 34 | SVTE | 257.25 | H | Ser | Val | Thr | Glu | | | | | | | |
| SEQ ID NO. 35 | SVTEQ | 284.32 | H | Ser | Val | Thr | Glu | Gln | | | | | | |
| SEQ ID NO. 36 | SVTEQG | 256.26 | H | Ser | Val | Thr | Glu | Gln | | Gly | | | | |
| SEQ ID NO. 37 | SVTEQGA | 315.33 | H | Ser | Val | Thr | Glu | | | | | Gln | Ala | OH |
| SEQ ID NO. 38 | EQGA | 301.30 | | | | | Glu | | | | | Gln | Ala | OH |
| SEQ ID NO. 39 | VTEQGA | 256.26 | | | | | Glu | | Thr | | Val | Gln | Ala | OH |
| SEQ ID NO. 40 | SVTQAA | 270.29 | H | Ser | Val | Thr | Gln | Ala | | Ala | | | | |

In some embodiments, the peptide is selected from the group consisting of SEQ ID NO. 35 to 39.

In some embodiments, the peptide is of 6 or 7 amino acid residues and comprises formulae (I) and (II), such as SEQ ID NO. 37.

The peptide may be capable of regulating leukocyte trafficking, such as inhibiting leukocyte migration. For example, the peptide may regulate, e.g. inhibit T lymphocyte, neutrophil, eosinophil, dendritic cell, basophil, monocyte and B lymphocyte migration. Of particular mention is the inhibition of T lymphocyte, neutrophil, eosinophil and dendritic cell migration.

The terms “T cell” and “T lymphocyte” are herein used interchangeably. The T lymphocyte migration is preferably trans-endothelial. The T lymphocytes may be auto-reactive T cells. The T cells may be CD4+ or CD8+.

The action of the peptide may be as an agonist of the already known PEPITEM receptor, CDH15.

It is understood that the peptide acts upon the individual to whom it is administered. As such, the auto-reactivity of any leukocytes such as T cells is reactivity against self from that individual. The individual may be a mammal, preferably a rodent such as a rat or mouse, or a primate, particularly an ape or human.

The peptide may act to regulate migration of leukocytes by several mechanisms, e.g. by regulating migration into and/or out of a tissue and/or by reducing the number of leukocytes by inducing apoptosis of leukocytes.

As the presence of the peptide, in some embodiments, serves to regulate the migration of the leukocytes, increasing the amount of peptide that the individual is exposed to serves to further regulate, e.g inhibit, said migration. The level of regulation of migration may be such that migration is reduced or inhibited by at least about 30% (in terms of numbers of leukocytes such as T cells that are recruited), at least about 40%, at least about 50%, at least about 60%, at least about 70%, least about 80%, at least about 90%, at least about 95%, or at least about 99%. In some embodiments the migration of the leukocytes is reduced to negligible levels.

In some embodiments, the peptide has an IC₅₀ value of less than about 0.15, less than about 0.10, less than about 0.05, less than about 0.03, less than about 0.02 or less than about 0.015 nM in a trans-migration assay. The IC₅₀ value may be determined using the methods described herein.

In some embodiments, the half-life (T_{1/2}) of the peptide in plasma is at least about 30 minutes, at least about 60 minutes, at least about 90 minutes, at least about 120 minutes, at least about 150 minutes or at least about 180 minutes. The half-life may be determined using the methods described herein.

In some embodiments, the half-life may be no greater than about 360 minutes, no greater than about 300 minutes, no greater than about 240 minutes or no greater than about 180 minutes. The half-life of the peptide may be the half-life in the plasma of a mammal. The mammal may be a rodent (e.g. mouse or rat), guinea pig, rabbit, dog, cat, pig, sheep, cow, primate (e.g. chimpanzee) or human.

Included within the scope of the invention are isotopically-labelled peptides. Isotopically-labelled peptides are identical to those recited herein, but for the fact that one or more atoms are replaced by an atom having an atomic mass or mass number different from the atomic mass or mass number predominantly found in nature. Examples of isotopes that can be incorporated into compounds disclosed herein include isotopes of hydrogen, carbon, nitrogen, oxygen and sulfur, such as ²H, ³H, ¹³C, ¹⁴C, ¹⁵N, ¹⁸O, ¹⁷O, and ³⁵S, respectively. It is well known in the art that isotopically labelling a compound often affects the half-life of said compound. The half-life of a compound is shorter when the compound is easily metabolised in its environment, for example by peptidases in plasma. More energy is required to break chemical bonds such as peptide bonds, when at least one of the atoms forming the bond, or one of the atoms close to the bond, is replaced with a heavier isotope. Thus, metabolism of a compound may be inhibited when at least one of the atoms of within the compound is replaced with a heavier isotope. For example, metabolism of a peptide is likely to be inhibited when one or more of the carbon atoms and/or nitrogen atoms that make up the peptide bond is replaced with a heaver isotope (e.g. ¹⁴C or ¹⁵N).

The peptides of the disclosure may exist in different stereoisomeric forms. All stereoisomeric forms and mixtures thereof, including enantiomers and racemic mixtures, are included within the scope of the invention. Such stereoisomeric forms include enantiomers and diastereoisomers. Individual stereoisomers of compounds disclosed herein, i.e., associated with less than 5%, preferably less than 2% and in particular less than 1% of the other stereoisomer, are included. Mixtures of stereoisomers in any proportion, for example a racemic mixture comprising substantially equal amounts of two enantiomers are also included within the invention.

Solvates of the peptides are also included, as well as amorphous and crystalline forms of the peptides.

Also included are pharmaceutically acceptable salts of the peptide, which may be prepared by treatment of the peptide with a suitable acid, such as hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, citric acid, benzoic acid and ascorbic acid.

Prodrugs of the peptides disclosed herein are also within the scope of the invention. Upon administration to a subject, a prodrug undergoes conversion by metabolic or other chemical processes to yield the peptide. Accordingly, in a further aspect, there is provided a molecule comprising the peptide.

Variants and derivatives of the peptide are included, wherein the peptide is modified, for example at the N- and/or C-termini and/or at the side chain of an amino acid residue. In some embodiments, the peptide is modified by glycosylation, wherein a glycan (polysaccharide) reacts with a glycosyl acceptor. The glycosyl acceptor is often any one or a combination selected from the group consisting of the NH₂ of an asparagine and arginine residue and the OH of a serine, threonine and tyrosine residue. In some embodiments, the peptide is modified by pegylation, wherein polyethylene glycol (PEG or macrogol) is bound to the peptide. In some embodiments, the peptide is modified with phosphorylcholine (ChoP), which comprises a negatively charged phosphate group bonded to a positively charged choline group. PEG and ChoP is often bound to the peptide via reaction with any one or a combination selected from the NH₃⁺ of a lysine residue; the SH of a cysteine residue; the NH of histidine; the NH and/or NH₂ of an arginine residue; the CO₂⁻ of an aspartic acid and glutamic acid residue; the NH₂ of an asparagine and arginine residue and the OH of a serine, threonine and tyrosine residue.

In some embodiments, the peptide is modified by linking it directly or indirectly to an immunoglobulin Fc (fragment crystallizable) region. The solubility of the peptides disclosed herein may be improved by linkage to an Fc region, which may improve the stability and/or half-life of the peptide. An Fc region or domain of an immunoglobulin molecule (also termed an Fc polypeptide) corresponds largely to the constant region of an immunoglobulin heavy chain, and is responsible for various functions, including an antibody's effector function(s). The Fc domain contains part or all of a hinge domain of an immunoglobulin molecule plus a CH2 and a CH3 domain. The Fc domain can form a dimer of two polypeptide chains joined by one or more disulfide bonds. In some embodiments, the Fc is a mammalian Fc such as a murine or human Fc. Various linkers are known in the art for linking a peptide to an immunoglobulin Fc region.

According to the fifth aspect, there is provided a peptide as defined in the first aspect, wherein the peptide is not of the sequence SVT (SEQ ID NO. 2). For the avoidance of doubt, each of the embodiments and clauses of the first aspect applies mutatis mutandis to the fifth aspect, with the proviso that the peptide is not of the sequence SVT. In some cases, the peptide of the fifth aspect does not comprise the sequence SVT.

Whilst it is possible for the peptide of the first and fifth aspects to be administered alone, it is typical to use a pharmaceutical composition. According to a sixth aspect, there is provided a pharmaceutical composition comprising a therapeutically effective amount of the peptide as defined herein and a pharmaceutically acceptable excipient.

The pharmaceutical composition may comprise one or more other drugs such as steroids, e.g. corticosteroids. In some embodiments, the pharmaceutical composition comprises a therapeutically effective amount of the peptide defined herein and a topical steroid. Alternatively, the pharmaceutical composition may comprise a steroid-sparing agent such as cyclosporin and/or lifitegrast. In some embodiments, the pharmaceutical composition comprises Rituximab.

The excipient may aid transport of a compound to the site in the body where it is intended to act, for example by increasing the rate of dissolution of the compound into the blood stream or by increasing the stability of the compound in order to delay its release, in order to increase its efficiency and prevent damage to tender tissues. Alternatively, the excipient may be for identification purposes, or to make the compound more appealing to the patient, for example by improving its taste, smell and/or appearance. Typically, the excipient makes up the bulk of the pharmaceutical composition.

Excipients include diluents or fillers, binders, disintegrants, lubricants, colouring agents and preservatives. Diluents or fillers are inert ingredients that may affect the chemical and physical properties of the final composition. If the dosage of the compound disclosed herein is small then more diluents will be required to produce a composition suitable for practical use. If the dosage of the compound disclosed herein is high then fewer diluents will be required.

Binders add cohesiveness to powders in order to form granules, which may form a tablet. The binder must also allow the tablet to disintegrate upon ingestion so that the compound dissolves. Disintegration of the composition after administration may be facilitated through the use of a disintegrant.

Any suitable pharmaceutically acceptable excipient is within the scope of the invention. Acceptable carriers, excipients, or stabilizers are nontoxic to recipients at the dosages and concentrations employed, and include buffers such as phosphate, citrate, and other organic acids; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cydohexanol, 3-pentanol, and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents to enhance preservative effectiveness such as EDTA, edetate salts, like edetate disodium, edetate calcium disodium, edetate sodium, edetate trisodium, and edetate dipotassium; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS®, or polyethylene glycol (PEG).

According to a further aspect, there is provided a dosage form comprising a pharmaceutical composition according to the sixth aspect. In a further aspect, there is provided a peptide, a pharmaceutical composition or a dosage form according to the present disclosure for use as a medicament or in therapy.

Pharmaceutical compositions can be formulated so as to allow a peptide according to the present disclosure to be bioavailable upon administration of the composition to an animal, such as a human. Compositions can take the form of one or more dosage units, where for example, a tablet can be a single dosage unit, and a container of a compound disclosed herein may contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms. The term “carrier” refers to a diluent, adjuvant or excipient, with which the peptide is administered. Suitable pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In some embodiments, when administered to an animal, the peptides and compositions disclosed herein, and pharmaceutically acceptable carriers are sterile. Preservatives may be used to prevent bacterial contamination in the compositions. Examples of preservatives are benzalkonium chloride, stabilized oxychloro complexes (otherwise known as Purite®), phenylmercuric acetate, chlorobutanol, benzyl alcohol, parabens, and thimerosal.

Water is a preferred carrier when the peptides disclosed herein are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

It is preferable that an effective amount of buffer be included to maintain the pH from about 6 to about 8, preferably about 7. Buffers used are those known to those skilled in the art, and, while not intending to be limiting, some examples are acetate, borate, carbonate, citrate, and phosphate buffers. Preferably, the buffer comprises borate. An effective amount of buffer can be readily determined by a person skilled in the art without undue experimentation. In cases where the buffer comprises borate, it is preferable that the concentration of the borate buffer be about 0.6%.

When intended for oral administration, the composition is preferably in solid or liquid form, where semi-solid, semi-liquid, suspension and gel forms are included within the forms considered herein as either solid or liquid.

The composition may comprise tonicity agents to adjust the concentration of dissolved material to the desired isotonic range. Tonicity agents are known to those skilled in the art; some examples include glycerin, mannitol, sorbitol, sodium chloride, and other electrolytes.

As a solid composition for oral administration, the composition can be formulated into a powder, granule, compressed tablet, pill, capsule, chewing gum, wafer or the like. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavouring agent such as peppermint, methyl salicylate or orange flavouring; and a colouring agent.

When the pharmaceutical composition is in the form of a capsule (e.g. a gelatin capsule), it can contain, in addition to materials of the above type, a liquid carrier such as polyethylene glycol, cyclodextrins or a fatty oil.

The pharmaceutical composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Surfactants may be used to help solubilize the peptide or one or more other components of the composition. Anionic, cationic, amphoteric, zwitterionic, and nonionic surfactants may all be used. Nonionic surfactants, such as polysorbates, poloxamers, alcohol ethoxylates, ethylene glycol-propylene glycol block copolymers, fatty acid amides, alkylphenol ethoxylates, or phospholipids may also be used.

For topical ocular administration, compositions comprising the peptides disclosed herein will be formulated as solutions, suspensions, emulsions and other dosage forms. Aqueous solutions are generally preferred, based on ease of formulation, as well as a patient's ability to administer such compositions easily by means of instilling one to two drops of the solutions in the affected eyes. However, the compositions may also be suspensions, viscous or semi-viscous gels, or other types of solid or semi-solid compositions. Suspensions may be preferred for compounds that are sparingly soluble in water.

An alternative for administration to the eye is intravitreal injection of a solution or suspension of the peptides disclosed herein. In addition, the peptides disclosed herein may also be introduced by means of ocular implants or inserts.

Examples of the administration form include without limitation oral, topical, parenteral, sublingual, rectal, vaginal, ocular and intranasal. Parenteral administration includes intravenous, intramuscular, intrathecal, intraosseous, subcutaneous, intravenous, intramuscular, intrasternal or infusion techniques. The peptide may be injected directly into a fracture, or administrated directly onto bone cells or surrounding media or by implant. In some embodiments, the peptide is administered by any one of the methods consisting of intravenous, intramuscular, intrathecal and subcutaneous administration, injection directly into a fracture, administration directly to the bone cells or surrounding media and administration by implant.

In some embodiments, the peptide is administered orally, for example in tablet form, or by injection.

Ocular administration includes topical administration to the eye, intraocular injection, for example intravitreal injection, and administration by means of ocular implants or inserts.

“Implant” is used herein to refer to any biocompatible device for insertion into the patient, and which releases the peptide into its surrounding area. Such devices are particularly useful for controlled and/or sustained peptide release. Effective amounts of peptide may be released from such a device for a period of several hours to several years. For a review of drug-releasing implants, see Santos, A. et al., J. Mater. Chem. B, 2014, 2, 6157-6182 and Stewart, S. A. et al., Polymers (Basel), 2018, 10 (12), 1379. The skilled person is aware that the rate of peptide release from an implant is dependent on the materials used to form the implant and the flow of bodily fluids surrounding the implant. For example, the implant may comprise membranes that are semi-permeable to the peptide and thus delay or decrease the rate of peptide release.

The amount of the peptide disclosed herein that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgement of the practitioner and each patient's circumstances.

The compositions comprise an effective amount of a compound disclosed herein such that a suitable dosage will be obtained. The correct dosage of the compounds will vary according to the particular formulation, the mode of application, and its particular site, host and the disease being treated. Other factors like age, body weight, sex, diet, time of administration, rate of excretion, condition of the host, drug combinations, reaction sensitivities and severity of the disease should be taken into account. Administration can be carried out continuously or periodically within the maximum tolerated dose.

Typically, the amount is at least about 0.01% of the peptide, and may comprise at least about 80%, by weight of the composition. When intended for oral administration, this amount can be varied to range from about 0.1% to about 80% by weight of the composition. Preferred oral compositions can comprise from about 4% to about 50% of the peptide by weight of the composition.

Pharmaceutical compositions disclosed herein may be prepared so that a parenteral dosage unit contains from about 0.01% to about 10% by weight of the peptide. In some embodiments a parenteral dosage unit contains about 0.5% to about 5% by weight of the peptide.

For intravenous administration, the composition is suitable for doses from about 0.1 mg/kg to about 250 mg/kg of the animal's body weight, preferably from about 0.1 mg/kg and about 20 mg/kg of the animal's body weight, and more preferably from about 1 mg/kg to about 10 mg/kg of the animal's body weight.

Generally, for compositions intended to be administered topically to the eye in the form of eye drops or eye ointments, the total amount of the peptides disclosed herein will be about 0.0001 to less than 5.0% (w/w). The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use.

The pharmaceutical compositions can be prepared using methodology well known in the art. For example, a composition intended to be administered by injection can be prepared by combining a compound disclosed herein with water, or other physiologically suitable diluent, such as phosphate buffered saline, so as to form a solution. A surfactant can be added to facilitate the formation of a homogeneous solution or suspension.

In the first aspect, the peptide or pharmaceutical composition disclosed herein is provided for use as a medicament.

Owing to their ability, in certain embodiments, to inhibit the migration of leukocytes, the peptides of the disclosure are useful in treating a number of conditions. These conditions include those in which leukocytes play a role in pathology or conditions associated with leukocyte migration, including inflammation such as immune mediated inflammatory diseases and/or musculoskeletal (MSK) loss and/or damage and/or allergic diseases. (see WO2018/165218A1; G. Chen et al., J. Clin. Invest., 2020, 19; Chimen et al., supra; J. M. Konter et al., J. Immunol., 2012, 188 (2), 854-863; H. Matsubara et al., J. Immunol., 2020, 204, 2043-2052; G. Schett et al., Nat. Rev. Immunol., 2020; J. R. Teijaro et al., Cell, 146, 980-991; H. Teoh et al., Am. J. Physiol. Endocrinol. Metab., 2008, 295 (3), E658-E664); M. Van Meurs, Shock, 2012, 37 (4), 392-398; K. B. Walsh et al., PNAS, 2011, 108 (29), 12018-12023; K. B. Walsh et al., J. Virol., 2014, 88 (11), 6281-93; Z. Xu et al., Lancet. Resp. Med., 2020, 8, 420-422; A. Marzano et al., Frontiers in Immunology, 2019, 10, 1069; and P. C. Fulkerson and M. E. Rothemberg, Nat. Rev. Drug Discov., 2013, 12 (2), 1-23). In particular, these conditions include any of the conditions treatable by administration of PEPTITEM including autoimmune and chronic inflammatory disease (Chimen et al., supra), dry eye and ocular diseases of inflammation (WO 2018/165218 (Allysta Pharmaceuticals, Inc.)), Systemic Lupus Erythematosus (H. Matsubara et al., J. Immunol., 2020, 204, 2043-2052), as well as diseases driven by eosinophils such as asthma, atopic dermatitis, allergic rhinitis, eosinophilic gastroenteritis, and certain eye diseases (P. C. Fulkerson and M. E. Rothernberg, Nat. Rev. Drug Discov., 2013, 12 (2), 1-23) and neutrophil driven disease such as neutrophil mediated skin disease, e.g. pyoderma gangrenosum (PG) and Sweet's syndrome (SS) (A. Marzano et al., Frontiers in Immunology, 2019, 10, 1069).

The peptides of the disclosure are also useful in reducing bone loss and/or stimulating bone production, particularly stimulating bone production (U.S. 62/912,439, supra).

Thus, according to a further aspect of the present disclosure, there is provided a peptide, a pharmaceutical composition or a dosage form disclosed herein for use in the treatment and/or prophylaxis of inflammation such as an immune-mediated inflammatory disease (IMID) and/or musculoskeletal (MSK) loss and/or damage

In some embodiments, the peptide, pharmaceutical composition or dosage form is for use in the treatment and/or prophylaxis of inflammation, such as immune-mediated inflammatory disease (IMID).

IMID is a group of conditions or diseases which are characterized by shared inflammatory pathways leading to an inappropriate or excessive inflammatory response that may cause pain, distress, loss of tissue and organ function and, in some cases, early death. IMIDs are clinically diverse, for example, rheumatoid arthritis (RA) affects joints whilst psoriasis targets skin, systemic lupus erythematosus (SLE) may affect the kidneys and the skin, as well as the heart, lungs and brain. Autoimmune hepatitis and primary biliary cirrhosis both cause damage to the liver and Sjögren's syndrome can stop tear and salivary glands working, meaning those affected have dry eyes and mouth amongst other symptoms. Despite these differences, many of these diseases share the similar genetic & environmental factors and inflammatory mechanisms. A cardinal feature of IMIDs is uncontrolled migration of T-cells into the affected organ where they release pro-inflammatory cytokines that drive a chronic and excessively vigorous inflammatory response. Indeed the cytokine profiles of diseases as disparate as rheumatoid arthritis (joint), type-1 diabetes (pancreas) and Crohn's disease (gastrointestinal tract) are known to share a common profile (Williams and Meyers, Immune-Mediated Inflammatory Disorders (I.M.I.D.s): The Economic and Clinical Costs; The American Journal of Managed Care, 2002).

Due to the commonality of the inflammatory process in IMIDs current standard of care therapies are often used across diverse indications. Indeed, regulating the traffic of T-cells into diseased tissues is an important mode of action of numerous pharmacological agents utilised to treat IMIDs.

The IMID may result in acute or chronic inflammation.

It is known that intraocular T cells play a significant role in ocular inflammatory disorders and therefore in some embodiments the IMID is an ocular inflammatory disorder selected from the group consisting of: dry eye disease, anterior and posterior uveitis (ocular disease), atopic keratoconjunctivitis, vernal keratoconjunctivitis, seasonal and perennial allergic conjunctivitis, eye inflammation post-surgery and laser treatment.

In some embodiments, the dry eye disease is selected from any one of the group consisting of hypolacrimation, tear deficiency, xerophthalmia, Sjogren's syndrome dry eye, non-Sjogren's syndrome dry eye, keroconjunctivitis sicca, aqueous tear-deficiency dry eye (ADDE), evaporative dry eye (EDE), environmental dry eye, Stevens-Johnson syndrome, ocular pemphigoid blepharitis marginal, eyelid-closure failure, sensory nerve paralysis, allergic conjunctivitis-associated dry eye, post-viral conjunctivitis dry eye, post-cataract surgery dry eye, VDT operation-associated dry eye, and contact lens wearing-associated dry eye.

The ocular inflammatory disorder is more particularly selected from the group consisting of: dry eye disease, anterior and posterior uveitis and atopic keratoconjuntivitis.

In some embodiments the IMID is selected from the group consisting of: inflammation caused by gene therapy vectors and other biologics, viral inflammation, systemic lupus erythematosus, virally induced T cell driven cytokine storm such as septicaemia, acute respiratory distress syndrome (ARDS), chronic obstructive pulmonary disease (COPD), lung fibrosis such as idiopathic pulmonary fibrosis (IPF), rheumatoid arthritis, psoriatic arthritis, Crohn's disease, inflammatory bowel disease (IBD), psoriasis, type I diabetes mellitus, multiple sclerosis, ulcerative colitis, systemic sclerosis, sinusitis, graft versus host disease, asthma, allergies, Sjogren's syndrome, photodermatitis, ankylosing spondylitis, lymphoid interstitial pneumonitis, Peyronie's disease, Behcet's disease, inflammatory and fibrotic liver disease(s) including steatohepatitis, autoimmune hepatitis and cirrhosis, sarcoidosis, giant cell arteritis, and ischaemia/reperfusion injury.

In some embodiments, the IMID is an inflammatory arthritis such as gout.

Without being bound by theory, it is believed that the ability of the peptides to inhibit the transmigration of T cells indicates their potential use in the treatment of IMIDs.

In some embodiments the IMID is selected from the group consisting of: dry eye disease, anterior and posterior uveitis (ocular disease), atopic keratoconjunctivitis, vernal keratoconjunctivitis, seasonal and perennial allergic conjunctivitis, eye inflammation post-surgery and laser treatment, inflammation caused by gene therapy vectors and other biologics, viral inflammation, systemic lupus erythematosus, virally induced T cell driven cytokine storm such as septicaemia, acute respiratory distress syndrome (ARDS), chronic obstructive pulmonary disease (COPD), and lung fibrosis such as idiopathic pulmonary fibrosis (IPF).

In some embodiments, the peptide, pharmaceutical composition or dosage form disclosed herein is for use in the treatment and/or prophylaxis of musculoskeletal (MSK) loss and/or damage.

Musculoskeletal loss and production are dependent on the balance of osteoclast and osteoblast activities. If the activities are such that the rate of bone cell generation is greater than the rate of bone cell resorption then there is an overall production of bone. If the rate of bone cell resorption is greater than the rate of bone cell generation then there is overall bone loss. On the other hand, if the rates of bone cell production and bone cell resorption are approximately equal then the amount of bone is approximately constant.

Bone is herein defined to be any type of bone tissue, i.e. cortical bone tissue, cancellous bone tissue and/or bone marrow. All of these tissues are formed by osteoblasts, which produce the protein osteoid, which mineralises to become bone.

Bone cells are defined to be any type of cell found in bone, including osteoblasts, osteoclasts and osteocytes. Osteocytes are derived from osteoblasts and contribute to bone regeneration by directing osteoblasts to sites in need of repair.

MSK diseases such as osteoporosis, in which bones weaken and become more brittle, reflect a relative enhancement of osteoclast activity such that the rate of bone cell absorption is greater than bone cell generation. Thus, osteoclasts are a prominent therapeutic target, and their inhibition or apoptosis is the mechanism of action of the commonly-used bisphosphonate MSK agents. However, long-term use of bisphosphonates is associated with increased incidence of micro-fractures and atypical femur fractures, suggesting that these drugs may hinder normal bone remodelling and repair. Furthermore, use of bisphosphonates in children has in some cases induced osteopetrosis, in which bones become abnormally dense and prone to fracture, see S. L. Teitelbaum, Am. J. Pathol., 2007, 170 (2), 427-435.

In contrast, the peptides of the invention stimulate the production of bone by osteoblasts, thereby increasing the rate of bone formation relative to bone absorption with the result that bone loss is reduced and, when the rate of bone formation is increased such that it is greater than the rate of bone resorption, bone is produced. Therefore, use of the peptides of the invention is not limited by the inherent and potentially under-active activity of the osteoblast cells that are treated.

The peptides of the invention have the potential to treat both inflammation and MSK damage simultaneously, providing an advantage over other therapies where either inflammation or MSK damage is treated by a drug. Such treatment may be especially beneficial where a patient is suffering from a condition such as rheumatoid arthritis, psoriatic arthritis, JIA, osteoarthritis and/or spondyloarthritis, where inflammation-induced bone damage may result in a need for joint replacement surgery. Inflammatory arthritis, such as gout, may also cause inflammation-induced bone damage. In some embodiments, musculoskeletal loss and/or damage is associated with osteoporosis (such as osteoporosis resulting from rheumatoid arthritis, psoriatic arthritis, juvenile idiopathic arthritis (JIA), and/or osteoarthritis) and/or bone injury. For a detailed review of the primary and secondary causes of osteoporosis, see Chapter 3: Diseases of Bone of the U.S. Department of Health and Human Services. Bone Health and Osteoporosis: A Report of the Surgeon General. Rockville, MD: U.S. Department of Health and Human Services, Office of the Surgeon General, 2004. In some embodiments, said osteoporosis results from any one or a combination of the group consisting of aging; prolonged bed rest; space travel; autoimmune diseases including rheumatoid arthritis, psoriatic arthritis, JIA, and osteoarthritis; genetic disorders including cystic fibrosis, Ehlers-Danlos, glycogen storage diseases, Gaucher's disease, homocystinuria, hypophosphatasia, idiopathic hypercalciuria, Marfan syndrome, Menkes steely hair syndrome, osteogenesis imperfect, porphyria and Riley-Day syndrome; hypogonadal states including androgen insensitivity, anorexia nervosa, athletic amenorrhea, hyperprolactinemia, panhypopituitarism, premature ovarian failure and Turner's and Klinefelter's syndrome; endocrine disorders including acromegaly, adrenal insufficiency, Cushing's syndrome, diabetes mellitus (Type 1), hyperparathyroidism and thyrotoxicosis; gastrointestinal diseases including gastrectomy, inflammatory bowel disease, malabsorption, celiac disease and primary biliary cirrhosis; hematologic disorders including haemophilia, leukemias and lymphomas, multiple myeloma, sickle cell disease, systemic mastocytosis and thalassemia; alcoholism; amyloidosis; chronic metabolic acidosis; congestive heart failure; depression; emphysema; end stage renal disease; epilepsy; idiopathic scoliosis; immobilisation; multiple sclerosis; muscular dystrophy; post-transplant bone disease; and sarcoidosis.

In some embodiments, said osteoporosis results from inflammation-induced bone damage. For example, said osteoporosis results from rheumatoid arthritis, psoriatic arthritis, JIA, and/or osteoarthritis. The osteoporosis may also result from inflammatory arthritis, such as gout.

In some embodiments, said osteoporosis results from any one or a combination of the group consisting of aging, prolonged bed rest, rheumatoid arthritis, psoriatic arthritis, osteoarthritis, anorexia nervosa, Diabetes Mellitus (Type 1), hyperparathyroidism, inflammatory bowel disease, malabsorption, celiac disease, haemophilia, leukemias and lymphomas, multiple myeloma, lupus, alcoholism, depression, emphysema, epilepsy, immobilisation, multiple sclerosis, muscular dystrophy and post-transplant bone disease.

In more specific embodiments, said osteoporosis results from any one or a combination of the group consisting of aging, prolonged bed rest, rheumatoid arthritis, psoriatic arthritis, osteoarthritis, anorexia nervosa, hyperparathyroidism, haemophilia, leukemias and lymphomas, multiple myeloma, alcoholism, depression, emphysema, epilepsy, immobilisation, muscular dystrophy and post-transplant bone disease.

In specific embodiments, the osteoporosis results from aging, i.e. is age-related osteoporosis. In other more specific embodiments, musculoskeletal loss and/or damage is associated with age-related osteoporosis.

In some embodiments, when musculoskeletal loss and/or damage is associated with osteoporosis, it is typically bone fracture or break. In other embodiments, musculoskeletal loss and/or damage is fracture, typically of the hip.

In some embodiments, the bone injury is associated with sports injuries, or is associated with any one or a combination of neurological disorders including stroke, multiple sclerosis, cerebral palsy, Parkinson's disease, spinal cord injury, neuropathy, sciatica and dementia; delirium; dizziness; vertigo; and dehydration.

In specific embodiments, the bone injury is break or fracture.

In a further aspect of the disclosure, there is provided the use of a peptide as defined herein in the manufacture of a medicament for the treatment and/or prophylaxis of inflammation such as immune-mediated inflammatory disease and/or musculoskeletal (MSK) loss and/or damage.

In another aspect, the disclosure teaches a method of treating a patient suffering from or at risk of inflammation such as immune-mediated inflammatory disease and/or musculoskeletal (MSK) loss and/or damage, the method comprising administering to the patient a therapeutically effective amount of a peptide, a pharmaceutical composition or a dosage form according to the present disclosure.

In a further aspect, the disclosure teaches a method of reducing bone loss and/or stimulating bone production, the method comprising administering an effective amount of a peptide disclosed herein, to a patient and/or bone cells or their precursors.

The bone cells comprise any one or a selection from the group consisting of osteoblasts, osteoclasts and osteocytes or their precursors. In some embodiments the bone cells consist primarily of osteoblasts and/or osteoblast precursors. In further embodiments, the bone cells are osteoblasts.

In some embodiments, the peptide is administered to a patient and/or bone cells. In these embodiments, the peptide is not administered to the precursors of the bone cells.

In one embodiment, the method is of stimulating bone production and comprises administering an effective amount of the peptide to a patient and/or bone cells and/or their precursors. In this embodiment, the rate of bone cell generation is greater than the rate of bone cell absorption such that there is an overall production of bone. Stimulating bone production may find use in dentistry and orthodontistry, in which any treatment requiring jaw bone growth and/or repair may benefit from application of an effective amount of the peptide of the invention. Such treatments include tooth and jaw alignment.

In specific embodiments, the peptide is administered ex vivo, i.e. in or on tissue in an external environment, outside of the patient. In such embodiments, the peptide is administered directly to bone cells and/or their precursors or surrounding media. Typically, the bone cells are osteoblasts. Often, the osteoblasts are primary osteoblasts, i.e. osteoblasts that are taken directly from living tissue and established for growth in vitro. In some embodiments the osteoblasts are derived from any one of the group consisting of a mammal, bird, reptile and amphibian. Typically, the osteoblasts are derived from a mammal. Preferably the osteoblasts are derived from a human.

In some embodiments, the peptide is administered ex vivo directly to the bone cells and/or their precursors or surrounding media. In some embodiments the peptide is administered directly to the bone cells or surrounding media, and not precursors of the bone cells. The bone cells may be primary bone cells derived from the patient. Alternatively, they may be derived from (their precursors may be) any one of the group consisting of mesenchymal stem cells, pluripotent stem cells, induced pluripotent stem cells, and peripheral blood mononuclear cells. Where the bone cells are derived from stem cells, they are typically derived from mesenchymal stem cells taken from bone marrow. For further information on the common types and sources of stem cells available, see Zakrzewski, W., Stem Cell. Res. Ther., 2019, 10 (68), 1-22. Bone cells may be prepared from stem cells in vitro by manipulating culture conditions, thereby restricting differentiation to specific pathways. For a review of in vitro directed differentiation see Cohen, D. E., Melton, D., Nat. Rev. Genet., 2011, 12, 243-252. The skilled person is aware of the conditions required to promote differentiation of stem cells to bone cells. Typically, osteoblasts are cultured in mineralisation differentiation media and osteoclasts are cultured in osteoclastogenic media.

Thus in a further aspect, there is provided a composition comprising the bone cells and/or their precursors and the peptide disclosed herein. In some embodiments, the composition does not comprise the precursors of the bone cells.

In certain embodiments, where the peptide is administered ex vivo onto the bone cells, the bone cells are then transplanted into a patient. The bone cells may be cultured for a specific time in vivo before transplant, or the bone cells may be transplanted immediately following ex vivo administration of the peptide. In such embodiments, the bone cells are transplanted conjunctly with the peptide. In other embodiments, the bone cells are transplanted consecutively or separately to the peptide.

The bone cells may be transplanted into the patient by any one or a combination of the methods consisting of intrathecal and intraosseous injection, injecting the cells directly into a fracture and administering the cells directly to the bone or surrounding media (for example, in open surgery). Bone formation by transplanted human osteoblasts has been reported by Yamanouchi, K. et al., J. Bone Miner. Res., 2001, 16 (5), 857-867 and accelerated bone fracture healing as a result of transplanted osteoblasts has been reported by Kim, S-J et al., BMC Musculoskelet. Disord., 2009, 10 (20), 1-9.

In some embodiments, where the peptide is administered ex vivo onto the bone cells, and the bone cells are then transplanted into a patient, the patient requires treatment and/or prophylaxis of musculoskeletal loss and/or damage.

Viewed from a further aspect, there is provided an orthopaedic implant comprising the peptide and in some embodiments peptide release from the orthopaedic implant is sustained for a period of several hours to several years.

The orthopaedic implant may or may not be biodegradable. Where the implant is biodegradable, it may be formed from one or a combination of the materials consisting of poly(lactic acid), poly(glycolic acid), poly(lactic-co-glycolic acid), poly(caprolactone), poly(amides), poly(anhydrides), poly(phosphazenes), poly(dioxanone), silk, cellulose and chitosan. Where the implant is non-biodegradable, it may be formed from one or a combination of the materials consisting of poly(siloxanes), poly(ethylene-vinyl acetate) and poly(urethanes).

The orthopaedic implant may comprise a polymer coating, and in some embodiments the implant comprises the peptide disclosed herein within a coating. The coating may be contacted with surgical hardware, including surgical plates, rods, pins, wires, washers, nails and screws that are typically used to repair bone damage (see Nguyen, V. D. and London, J. Radiology, 1986, 158, 129-131). Thus, in some embodiments, the orthopaedic implant comprises orthopaedic hardware coated with the peptide. Such an implant may achieve a sustained release of the peptide in a localised area of bone damage. For a review of bioactive coatings of orthopaedic implants, see Zhang, B. G. X et al., Int. J. Mol. Sci., 2014, 15 (7), 11878-11921 and Goodman, S. B., Keeney, Y. Z. and Yang F., Biomaterials, 2013, 34 (13), 3174-3183.

Commonly, orthopaedic implants are fixed into place using bone cement. Thus, viewed from a further aspect, there is provided a composition comprising bone cement and the peptide of the invention. In some embodiments, the bone cement is selected from any one of the group consisting of polymethyl methacrylate, calcium phosphate cement and glass polyalkenoate cement. For a review on bone cement, see Vaishya, R., Chauhan, M. and Vaish, A., J. Clin. Orthop. Trauma, 2013, 4 (4), 157-163.

When the peptide is administered by implant, it is typically administered as a single dose. However, replacement of the implant and further dose administration are included within the scope of the disclosure. When the peptide is administered by other means, it may be administered in one or more doses per one or more day(s). For example, the peptide may be administered in a single dose every day, week, fortnight or month, with the dose reducing or stopping on recovery of the bone.

The patient referred to herein may be any animal. In some embodiments, the patient is any one of the group consisting of mammal, bird, reptile and amphibian. In other embodiments, the patient is a mammal. In some embodiments, the patient is any one selected from the group consisting of human, horse, dog, cattle, goat, sheep, pig, cat, bison, camel, llama and alpaca. In more specific embodiments, the patient is any one selected from the group consisting of human, horse, dog, cattle, goat, sheep, pig and cat, most often a human.

It is to be understood that, where the patient is suffering from or at risk of musculoskeletal (MSK) loss and/or damage, the patient comprises a skeleton made of bone.

The peptide, pharmaceutical composition or dosage form may be administered in combination with one or more other active agents, such as anti-inflammatory agents. Thus, also provided is a combination of a peptide, pharmaceutical composition or dosage form disclosed herein, and one or more anti-inflammatory agents.

In some embodiments the peptide, pharmaceutical composition or dosage form according to the disclosure is administered topically.

In some embodiments, the anti-inflammatory agent is a non-steroidal anti-inflammatory drug, such as aspirin, diclofenac, ibuprofen, celecoxib, mefenamic acid, etoricoxib, or indomethacin.

In some embodiments the anti-inflammatory agent is a corticosteroid, for example prednisone, cortisone, or methylprednisone.

The peptide, pharmaceutical composition or dosage form and the other active agents may be administered simultaneously, separately or sequentially.

It will be appreciated that any of the embodiments described herein in relation to the first aspect may be combined with any other aspect, unless otherwise stated.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention without departing from the scope of the invention. The present embodiments are therefore to be considered for descriptive purposes. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The aspects and embodiments of this disclosure are further described in the following clauses:
1. A peptide of 3-7 amino acid residues for use as a medicament wherein the peptide comprises formula (I) and/or formula (II); wherein formula (I) is:

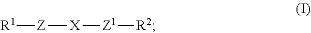

wherein Z and Z¹ are each independently an amino acid selected from serine and threonine; X is an amino acid selected from valine, leucine, phenylalanine, tryptophan and tyrosine; R¹ is hydrogen (H), COR³ or a bond to another amino acid; R² is OH, N(R⁴)₂ or a bond to another amino acid; and each R³ and R⁴ is independently selected from H and a C₁-C₆ alkyl group; and
formula (II) is:

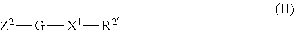

wherein Z² is selected from N, Q, Ac—N, Ac-Q or pyroglutamic acid, wherein Q represents glutamine and N represents asparagine; G represents glycine; X¹ is an amino acid selected from alanine, valine, leucine, phenylalanine and 2-amino-2-methylpropanoic acid; R^{2′} is OH, N(R^{4′})₂ or a bond to another amino acid; and each R⁴ is independently selected from H and a C₁-C₆ alkyl group; and
wherein the peptide is not of the sequence CSVTCG.
2. The peptide for use of clause 1, wherein when the peptide comprises formulae (I) and (II), it is represented by any one of formulae (IIIa) to (IIIf), (IVa) and (IVb):

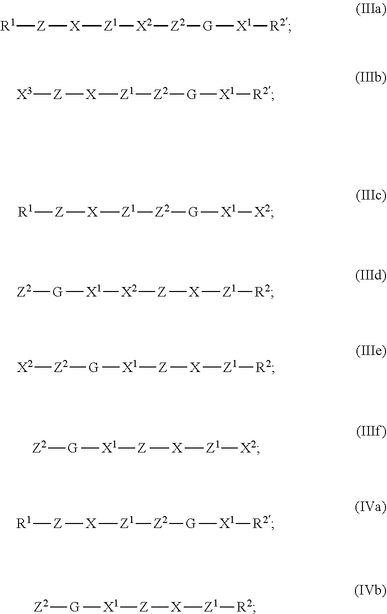

wherein X² and X³ are each an amino acid; R¹ is H or COR³; R^{2′} is OH or N(R⁴)₂; R² is OH or N(R⁴)₂; Z² of formulae (IIIa) to (IIIc), (IIIe) and (IVa) is N or Q; and Z² of formulae (IIId), (IIIf) and (IVb) is Ac—N, Ac-Q or pyroglutamic acid.
3. The peptide for use of clause 1, wherein when the peptide comprises formula (I) or formula (II), it is represented by any one of formulae (I), (II), (Va) to (Vd), (VIa to VIf), (VIIa) to (VIIh) and (VIIIa) to (VIIIh):

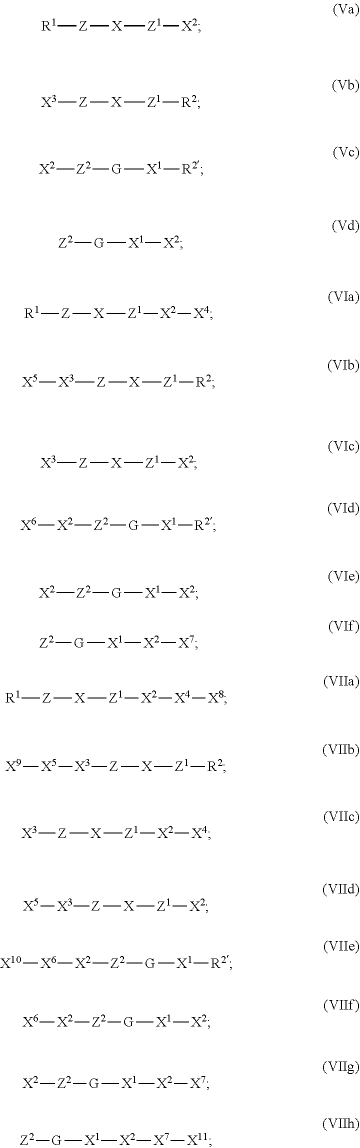

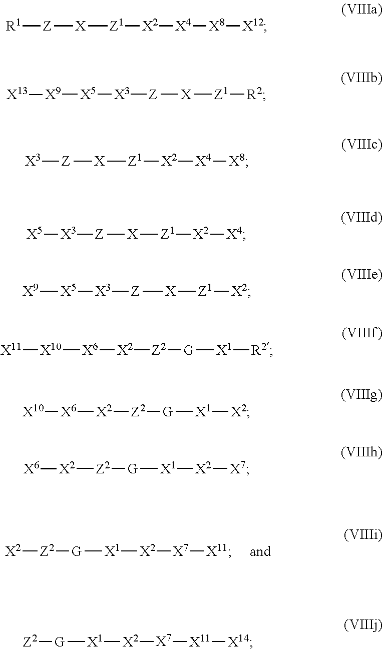

wherein X² to X¹⁴ are each an amino acid; R¹ is H or COR³; R² is OH or N(R⁴)₂; R^{2′} is OH or N(R^{4′})₂; Z² of formulae (Vc), (VId), (VIe), (VIIe) to (VIIg) and (VIIIf) to (VIIIi) is N or Q; and Z² of formulae (II), (Vd), (VIf), (VIIh) and (VIIIj) is Ac—N, Ac-Q or pyroglutamic acid.
4. The peptide for use of clause 3, wherein the peptide is represented by any one of formulae (I), (II), (Va) to (Vd), (VIa), (VIc) to (VIf), (VIIc), (VIIe) to (VIIh), (VIIIa) to (VIIIh), (IIIa) to (IIIf), (IVa) and (IVb).
5. The peptide for use of clause 3 or clause 4, wherein when the peptide is represented by formula (VIIc), X³ is an amino acid other than cysteine, X² is an amino acid other than cysteine and/or X⁴ is an amino acid other than glycine.
6. The peptide for use of any one of clauses 3 to 5, wherein when the peptide is represented by formula (VIIc), X² is glutamic acid and/or X⁴ is glutamine.
7. The peptide for use of any one of clauses 3 to 6, wherein when the peptide is represented by formulae (VIIIc) or (VIIId), X³ is an amino acid other than cysteine, X² is an amino acid other than cysteine and/or X⁴ is an amino acid other than glycine.
8. The peptide for use of any one of clauses 3 to 7, wherein when the peptide is represented by formula (VIIIc) or (VIIId), X² is glutamic acid and/or X⁴ is glutamine.
9. The peptide for use of any one of clauses 2 to 8, wherein X³ is an amino acid other than cysteine, X² is an amino acid other than cysteine and/or X⁴ is an amino acid other than glycine.
10. The peptide for use of any one of clauses 2 to 9, wherein X² is glutamic acid and/or X⁴ is glutamine.
11. The peptide for use of any one of any one of clauses 3 to 10, wherein X² is glutamic acid or aspartic acid; X⁴ is glutamine or asparagine; X⁶ is threonine or serine; X⁷ is any one selected from the group consisting of leucine, isoleucine, valine, alanine, methionine, phenylalanine, tyrosine and tryptophan, such as leucine, isoleucine, valine or alanine; X⁸ is glycine; X¹⁰ is any one selected from the group consisting of valine, leucine, phenylalanine, tryptophan and tyrosine, such as valine, leucine or phenylalanine; X¹¹ is serine or threonine; X¹² is any one selected from the group consisting of alanine, valine, leucine, phenylalanine and 2-amino-2-methylpropanoic acid; and X¹⁴ is asparagine or glutamine.
12. The peptide for use of any one of clauses 3 to 11, wherein X² is glutamic acid; X⁴ is glutamine; X⁶ is threonine; X⁷ is leucine; X⁸ is glycine; X¹⁰ is valine; X¹¹ is serine; X¹² is alanine; and X¹⁴ is asparagine.
13. The peptide for the use of any one of clauses 1 to 12, wherein X is any one selected from valine, leucine, phenylalanine and tryptophan.
14. The peptide for use of any one of clauses 1 to 12, wherein Z is serine and X is any one selected from valine, tryptophan and tyrosine.
15. The peptide for use of any one of clauses 1 to 12, wherein Z is serine and X is valine or tryptophan.
16. The peptide for use of any one of clauses 1 to 12, wherein Z—X—Z¹ is selected from SVT; SLT; SFT; SWT; SYT; TVT; SVS; and TVS.
17. The peptide for use of any one of clauses 1 to 12, wherein Z—X—Z¹ is selected from SVT; SLT; SFT; SWT; TVT; SVS; and TVS.
18. The peptide for use of any one of clauses 1 to 17, wherein X¹ is an amino acid selected from alanine, valine, leucine and phenylalanine.
19. The peptide for use of any one of clauses 1 to 17, wherein X¹ is an amino acid selected from alanine and leucine.
20. The peptide for use of any one of clauses 1 to 17, wherein Z²-G-X¹ is any one selected from QGA; NGA; Ac-QGA; pEGA; Ac-QGAib; Ac-NGA; pEGV; pEGL; and pEGF.
21. The peptide for use of any one of clauses 1 to 20, wherein R³ is a C₁₋₄alkyl group.
22. The peptide for use of any one of clauses 1 to 20, wherein R¹ is any one selected from H, COCH₃ and a bond to another amino acid.
23. The peptide for use of any one of clauses 1 to 22, wherein R⁴ is independently selected from H and a C₁₋₄alkyl group.
24. The peptide for use of any one of clauses 1 to 22, wherein R⁴ is independently selected from any one of the group consisting of H, methyl and ethyl.
25. The peptide for use of any one of clauses 1 to 22, wherein N(R⁴)₂ is NH₂, NH(CH₃) or NH(CH₂CH₃).
26. The peptide for use of any one of clauses 1 to 25, wherein R^{4′} is independently selected from H and a C₁₋₄alkyl group.
27. The peptide for use of any one of clauses 1 to 25, wherein R^{4′} is independently selected from any one of the group consisting of H, methyl and ethyl.
28. The peptide for use of any one of clauses 1 to 25, wherein N(R^{4′})₂ is NH₂, N(CH₃)₂, NH(CH₃) or NH(CH₂CH₃).
29. The peptide for use of any one of clauses 1 to 25, wherein R^{2′} is NH₂, or N(CH₃)₂.
30. The peptide for use of any one of clauses 1 to 29, comprising formula (I).
31. The peptide for use of any one of clauses 1 to 29, comprising formula (II).
32. The peptide for use of any one of clauses 1 to 29, comprising formula (I) and formula (II).
33. The peptide for use of clause 32, wherein the peptide is represented by any one of formulae (IIIa) to (IIIc) and (IVa).
34. The peptide for use of clause 32, wherein the peptide is represented by formulae (IIIa) or (IVa).
35. The peptide for use of clause 32, wherein the peptide is represented by formula (IIIa).
36. The peptide for use of clause 32, wherein the peptide is of SEQ ID NO. 37.
37 The peptide for use of any one of clauses 1 to 29, comprising formula (I) or formula (II).
38 The peptide for use of any one of clauses 1 to 37, wherein the peptide is a 3mer or 4mer.
39. The peptide for use of clause 37, wherein the peptide is a 3mer.
40. The peptide for use of clause 39, wherein the peptide is of formula (I).
41. The peptide for use of clause 39 or 40, wherein R¹ is H or COCH₃ and R² is NH₂ or NHR⁴, e.g. NH (ethyl).
42. The peptide for use of clause 40, wherein the peptide is of any one sequence selected from the group consisting of SEQ ID NO. 2 to 18.
43. The peptide for use of clause 40, wherein the peptide is of any one sequence selected from the group consisting of SEQ ID NO. 2 to 6, 8, 9, 11, and 15 to 18.
44. The peptide for use of clause 40, wherein the peptide is of any one sequence selected from the group consisting of SEQ ID NO. 4, 5, 9, 11 and 17.
45. The peptide for use of clause 39, wherein the peptide is of formula (II).
46. The peptide for use of clause 39 or clause 45, wherein Z² is any one selected from the group consisting of Ac—N, Ac-Q or pyroglutamic acid and R^{2′} is selected from NH₂, and N(CH₃)₂.
47. The peptide for use of clause 45, wherein the peptide is of any one sequence selected from the group consisting of SEQ ID NO. 19 to 33.
48. The peptide for use of clause 45, wherein the peptide is of any one sequence selected from the group consisting of SEQ ID NO. 19 to 23, 25, 26, and 28 to 33.
49. The peptide for use of clause 45, wherein the peptide is of any one sequence selected from the group consisting of SEQ ID NO. 23, 26, 28, 31 and 32.
50. The peptide for use of clause 37, wherein the peptide comprises 4 to 7 amino acids.
51. The peptide for use of clause 50, wherein the peptide is represented by any one of formulae (Va), (Vc), (Vd), (VIa), (VId) to (VIf), (VIIa), (VIIe) to (VIIh), (VIIIa), and (VIIIf) to (VIIIj).
52. The peptide for use of clause 50, wherein the peptide is represented by any one of formulae (Va), (Vc), (Vd), (VIa), (VId) to (VIf), (VIIe) to (VIIh), (VIIIa), and (VIIIf) to (VIIIj).
53. The peptide for use of clause 50, wherein the peptide is of any one sequence selected from the group consisting of SEQ ID NO. 34 to 36 and 38 to 40.
54. The peptide for use of clause 50, wherein the peptide is of any one sequence selected from the group consisting of SEQ ID NO. 35, 36, 38 and 39.
55. A peptide as defined in any one preceding clause for use in regulating leukocyte migration.
56. The peptide for use of clause 55, wherein the use is in inhibiting leukocyte migration and the level of inhibition of migration is such that migration is reduced by at least about 30%.
57. The peptide for use of clause 55 or clause 56, wherein the migration of the leukocytes is trans-endothelial.
58. A peptide as defined in any one of clauses 1 to 54, for use in the treatment and/or prophylaxis of inflammation and/or musculoskeletal loss and/or damage.
59. The peptide for use of claim 58, wherein the inflammation is a symptom of an immune mediated inflammatory disease, allergic disease or neutrophil mediated disease.
60. The peptide for use of claim 58, wherein the inflammation is a symptom of an immune mediated inflammatory disease.
61. The peptide for use of clause 59 or clause 60, wherein the immune-mediated inflammatory disease is selected from the group consisting of: dry eye disease, anterior and posterior uveitis (ocular disease), atopic keratoconjunctivitis, vernal keratoconjunctivitis, seasonal and perennial allergic conjunctivitis, eye inflammation post-surgery and laser treatment, inflammation caused by gene therapy vectors and other biologics, viral inflammation, systemic lupus erythematosus, virally induced T cell driven cytokine storm such as septicaemia, acute respiratory distress syndrome (ARDS), chronic obstructive pulmonary disease (COPD), lung fibrosis such as idiopathic pulmonary fibrosis (IPF), rheumatoid arthritis, psoriatic arthritis, Crohn's disease, inflammatory bowel disease (IBD), psoriasis, systemic lupus erythematosus, type I diabetes mellitus, multiple sclerosis, ulcerative colitis, systemic sclerosis, sinusitis, graft versus host disease, asthma, allergies, Sjogren's syndrome, photodermatitis, ankylosing spondylitis, lymphoid interstitial pneumonitis, Peyronie's disease, Behcet's disease, inflammatory and fibrotic liver disease(s) including steatohepatitis, autoimmune hepatitis and cirrhosis, sarcoidosis, giant cell arteritis, uveitis (ocular disease), septicaemia and ischaemia/reperfusion injury.
62. The peptide for use of clause 59 or clause 60, wherein the immune-mediated inflammatory disease is selected from the group consisting of: dry eye disease, anterior and posterior uveitis (ocular disease), atopic keratoconjunctivitis, vernal keratoconjunctivitis, seasonal and perennial allergic conjunctivitis, eye inflammation post-surgery and laser treatment, inflammation caused by gene therapy vectors and other biologics, viral inflammation, systemic lupus erythematosus, virally induced T cell driven cytokine storm such as septicaemia, acute respiratory distress syndrome (ARDS), chronic obstructive pulmonary disease (COPD), lung fibrosis such as idiopathic pulmonary fibrosis (IPF), rheumatoid arthritis, inflammatory arthritis such as gout, psoriatic arthritis, Crohn's disease, inflammatory bowel disease (IBD), psoriasis, systemic lupus erythematosus, type I diabetes mellitus, multiple sclerosis, ulcerative colitis, systemic sclerosis, sinusitis, graft versus host disease, asthma, allergies, Sjogren's syndrome, photodermatitis, ankylosing spondylitis, lymphoid interstitial pneumonitis, Peyronie's disease, Behcet's disease, inflammatory and fibrotic liver disease(s) including steatohepatitis, autoimmune hepatitis and cirrhosis, sarcoidosis, giant cell arteritis, uveitis (ocular disease), septicaemia and ischaemia/reperfusion injury.
63. The peptide for use of clause 61 or clause 62, wherein the dry eye disease is selected from any one of the group consisting of hypolacrimation, tear deficiency, xerophthalmia, Sjogren's syndrome dry eye, non-Sjogren's syndrome dry eye, keroconjunctivitis sicca, aqueous tear-deficiency dry eye (ADDE), evaporative dry eye (EDE), environmental dry eye, Stevens-Johnson syndrome, ocular pemphigoid blepharitis marginal, eyelid-closure failure, sensory nerve paralysis, allergic conjunctivitis-associated dry eye, post-viral conjunctivitis dry eye, post-cataract surgery dry eye, VDT operation-associated dry eye, and contact lens wearing-associated dry eye.
64. The peptide for use of clauses 58 to 63, wherein the musculoskeletal loss and/or damage is associated with osteoporosis and/or bone injury.
65. The peptide for use of clause 64, wherein the osteoporosis results from any one or a combination of the group consisting of aging; prolonged bed rest; space travel; autoimmune disorders including rheumatoid arthritis, psoriatic arthritis, osteoarthritis and JIA; genetic disorders including cystic fibrosis, Ehlers-Danlos, glycogen storage diseases, Gaucher's disease, homocystinuria, hypophosphatasia, idiopathic hypercalciuria, Marfan syndrome, Menkes steely hair syndrome, osteogenesis imperfect, porphyria and Riley-Day syndrome; hypogonadal states including androgen insensitivity, anorexia nervosa, athletic amenorrhea, hyperprolactinemia, panhypopituitarism, premature ovarian failure and Turner's and Klinefelter's syndrome; endocrine disorders including acromegaly, adrenal insufficiency, Cushing's Syndrome, Diabetes Mellitus (Type 1), hyperparathyroidism and thyrotoxicosis; gastrointestinal diseases including gastrectomy, inflammatory bowel disease, malabsorption, celiac disease and primary biliary cirrhosis; hematologic disorders including haemophilia, leukemias and lymphomas, multiple myeloma, sickle cell disease, systemic mastocytosis and thalassemia; rheumatic and auto-immune diseases including ankylosing spondylitis, lupus and rheumatoid arthritis; alcoholism; amyloidosis; chronic metabolic acidosis; congestive heart failure; depression; emphysema; end stage renal disease; epilepsy; idiopathic scoliosis; immobilisation; multiple sclerosis; muscular dystrophy; post-transplant bone disease; and sarcoidosis.
66. The peptide for use of clause 64, wherein the osteoporosis results from any one or a combination of the group consisting of aging; prolonged bed rest; space travel; autoimmune disorders including rheumatoid arthritis, inflammatory arthritis such as gout, psoriatic arthritis, osteoarthritis and JIA; genetic disorders including cystic fibrosis, Ehlers-Danlos, glycogen storage diseases, Gaucher's disease, homocystinuria, hypophosphatasia, idiopathic hypercalciuria, Marfan syndrome, Menkes steely hair syndrome, osteogenesis imperfect, porphyria and Riley-Day syndrome; hypogonadal states including androgen insensitivity, anorexia nervosa, athletic amenorrhea, hyperprolactinemia, panhypopituitarism, premature ovarian failure and Turner's and Klinefelter's syndrome; endocrine disorders including acromegaly, adrenal insufficiency, Cushing's Syndrome, Diabetes Mellitus (Type 1), hyperparathyroidism and thyrotoxicosis; gastrointestinal diseases including gastrectomy, inflammatory bowel disease, malabsorption, celiac disease and primary biliary cirrhosis; hematologic disorders including haemophilia, leukemias and lymphomas, multiple myeloma, sickle cell disease, systemic mastocytosis and thalassemia; rheumatic and auto-immune diseases including ankylosing spondylitis, lupus and rheumatoid arthritis; alcoholism; amyloidosis; chronic metabolic acidosis; congestive heart failure; depression; emphysema; end stage renal disease; epilepsy; idiopathic scoliosis; immobilisation; multiple sclerosis; muscular dystrophy; post-transplant bone disease; and sarcoidosis.
67. The peptide for use of clause 64, wherein the osteoporosis results from any one or a combination of the group consisting of aging, prolonged bed rest, rheumatoid arthritis, psoriatic arthritis, osteoarthritis, JIA, anorexia nervosa, Diabetes Mellitus (Type 1), hyperparathyroidism, inflammatory bowel disease, malabsorption, celiac disease, haemophilia, leukaemia's and lymphomas, multiple myeloma, lupus, rheumatoid arthritis, alcoholism, depression, emphysema, epilepsy, immobilisation, multiple sclerosis, muscular dystrophy and post-transplant bone disease.
68. The peptide for use of clause 64, wherein the osteoporosis results from any one or a combination of the group consisting of aging, prolonged bed rest, rheumatoid arthritis, psoriatic arthritis, osteoarthritis, JIA, anorexia nervosa, hyperparathyroidism, haemophilia, leukaemia's and lymphomas, multiple myeloma, alcoholism, depression, emphysema, epilepsy, immobilisation, muscular dystrophy and post-transplant bone disease
69. The peptide for use of clause 64, wherein the osteoporosis results from aging.
70. The peptide for use of any one of clauses 64 to 69, wherein the bone injury is associated with sports injuries or any one or a combination of neurological disorders including stroke, multiple sclerosis, cerebral palsy, Parkinson's disease, spinal cord injury, neuropathy, sciatica and dementia; delirium; dizziness; vertigo; and dehydration.
71. The peptide for use of any one of clauses 58 to 63, wherein the musculoskeletal loss and/or damage is associated with age-related osteoporosis.
72. The peptide for use of any one of clauses 58 to 63, wherein the musculoskeletal loss and/or damage is bone fracture.
73. A method of reducing bone loss and/or stimulating bone production, the method comprising administering an effective amount of the peptide defined in any one of clauses 1 to 54 to a patient and/or bone cells and/or their precursors.
74. The method of clause 73 wherein the method is of stimulating bone production.
75. The method of clause 73 or clause 74 wherein the peptide is administered ex vivo directly to the bone cells or their precursors or surrounding media.
76. The method of any one of clauses 73 to 75 wherein the bone cells are osteoblasts or osteoblast precursors.
77. The method of any one of clauses 73 to 76 wherein the bone cells are osteoblasts.
78 The method of clause 77 wherein the osteoblasts are primary osteoblasts.
79. The method of clause 77 or clause 78 wherein the osteoblasts are mammal osteoblasts.
80 The method of clause 77 or clause 78 wherein the osteoblasts are human osteoblasts.
81. The method of any one of clauses 75 to 80 further comprising transplanting the bone cells into a patient.
82. The method of clause 81 wherein the patient requires treatment and/or prophylaxis of musculoskeletal loss and/or damage.
83. The peptide for use of any one clauses 1 to 72, wherein the peptide has a half-life in the plasma of a mammal of at least about 60 minutes.
84. A peptide as defined in any one of clauses 1 to 54, wherein the peptide is not of the sequence SVT.
85. A pharmaceutical composition comprising a therapeutically effective amount of the peptide defined in any one of clauses 1 to 54 and 84 and a pharmaceutically acceptable excipient.

### EXAMPLES

The following non-limiting examples below serve to illustrate the invention further.

### Method of Synthesis

The peptide is optionally an isolated peptide. The peptide may be synthesized by any manner known in the art. Methods of synthesis include, but are not limited to, solid-phase synthesis, aqueous phase synthesis, enzymatic synthesis and recombinant synthesis.

The peptides may be synthesised by solid-phase synthesis using methods well known to those skilled in the art. Generally, the C-terminal end of an amino acid (with protected N-terminus) is attached to an appropriate solid support via the α-carboxyl group. The N-terminus is protected by an appropriate protecting group (such as tert-butyloxycarbonyl [Boc] or 9-fluorenylmethoxycarbonyl [Fmoc]). The solid support may be a resin such as a copolymer of styrene and 1% divinylbenzene. The N α-protecting group (e.g. Boc or Fmoc) is removed, and the N-terminal of the amino acid attached to the solid support is coupled to another amino acid using appropriate coupling reagents (such as dicyclohexylcarbodiimide). The dipeptide is elongated by repeating the deprotection and coupling steps. When each of the amino acids making up the desired peptide have been coupled together, any side-chain protecting groups used during the synthesis are removed, and the peptide is cleaved from the solid support. Acetic anhydride may be used to acetylate the N-terminal. If a C-terminal amide is preferable, an appropriate amide-containing resin is chosen such that when the peptide is cleaved from the resin, the amide group is retained on the C-terminus of the peptide. Common solid supports for the synthesis of peptides with C-terminal amides are benzhydrylamide derivatives, such as 4-methylbenzhydrylamine resin. The peptide amide can be cleaved from the resin using hydrogen fluoride.

Alternatively, the peptides may be prepared by recombinant synthesis. A nucleic acid sequence encoding the peptide can be cloned into an appropriate expression vector. The vector may then be introduced into a host cell for expression of the cloned sequence and production of the peptide. The nucleic acid sequence may be codon optimized for the particular host. The host may be a microorganism such as a bacterium or fungus. The peptide may be made as a fusion protein to facilitate expression/production or aid in peptide delivery to target. Following purification of the peptide, the N- and/or C-terminal groups may be modified by appropriate methods.

The peptides may be purified by conventional methods known in the art. These include the use of gel filtration and reverse-phase chromatography.

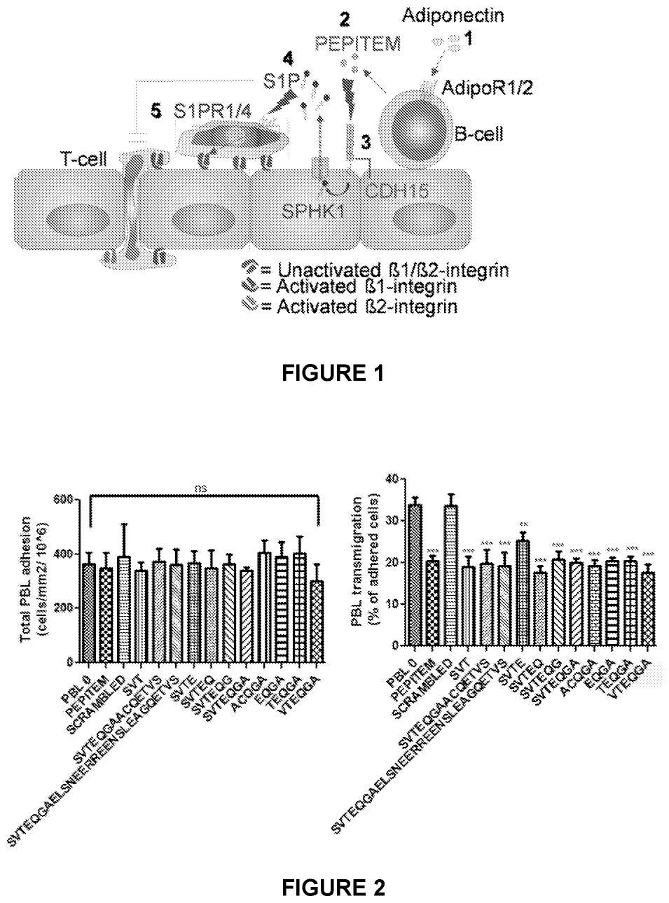

As described above, PEPITEM has been reported to be efficacious against T-cell trafficking via interactions with CDH-15 and in reducing bone loss and/or stimulating bone growth. PEPITEM functions as a master switch in the inflammatory response which is essential for controlling T-cell trafficking during inflammation (Chimen et al., supra). With reference to FIG. 1, B-lymphocytes (B-cells) are stimulated by adiponectin (1) and release PEPITEM, which is a 14 amino acid (AA) proteolytic cleavage product of the 14.3.3.3 protein, with the sequence SVTEQGAELSNEER (2) (SEQ ID NO. 1). PEPITEM stimulates endothelial cells through a membrane borne receptor, CDH15; M-cadherin (3), to release the bioactive phospholipid, sphingosine-1 phosphate (S1P) (4). S1P in turn down-regulates integrin adhesion receptors on T-cells, thereby imposing a tonic inhibition on their migration (5). Importantly, the trafficking of other circulating populations of leukocytes is unaffected by PEPITEM, including effector cells of the innate immune response such as neutrophils and monocytes. This indicates that PEPITEM is able to specifically modulate transmigration of memory T cells and Treg (WO 2013/104928).

PEPITEM is reported to be effective in reducing bone loss and/or stimulating bone growth by stimulating the activity of osteoblasts to trigger bone production whilst simultaneously inhibiting the activity of osteoclasts to limit bone loss.

### Materials and Methods

### General Protocol for Synthesis of the Peptides

Peptides were assembled manually on a 0.3 mmol scale by Fmoc (Fluorenylmethyloxycarbonyl)/t-Bu chemistry using the solid phase method (SPPS). Rink Amide AM resin (Novabiochem) was used to assemble C-terminal amide peptides. Pre-loaded Wang or 2-chlorotrityl chloride resin (Novabiochem) was used to assemble C-terminal acid peptides.

Couplings were generally achieved using 5 equivalents of Fmoc protected amino acid (Novabiochem) with PyBOP/DIPEA (5/10 equivalents) in dimethylformamide for 1 h. Polar peptides were prepared with N-terminal Boc (t-butyloxycarbonyl) groups on chlorotrityl resin and cleaved using 20% trifluoroethanol in dichloromethane (DCM) (3×1 h). Following RP-HPLC purification, the Boc group was removed using 30% TFA (Trifluoroacetic acid) in DCM and the peptide was lyophilised.

All peptide crudes were purified by reverse-phase HPLC using a C18 preparative column at 230 nm and 0.1% TFA containing buffers (A: milliQ water-B: acetonitrile). Analysis was performed on an Agilent 1100 HPLC using an ACE C18 column (150×2.1-300A-2 u). Molecular weights were confirmed by electrospray ionisation on a Waters Alliance HPLC equipped with a Micromass LCT detector.

### PBL Isolation

Blood samples were obtained from consensual healthy donors and were collected in EDTA tubes (Sigma-Aldrich). Peripheral blood mononuclear cells (PBMCs) were isolated using a two-step density gradient created using 2.5 ml of Histopaque 1077 layered on 2.5 ml of Histopaque 1119 (Sigma-Aldrich) in a 10 ml centrifuge tube. 5 ml of blood was layered on top of the density gradient, and was centrifuged at 2500 rpm for 45 minutes. The top layer containing the peripheral blood mononuclear cells (PBMCs) was isolated and made up to a volume of 12 ml with M199 (Gibco Invitrogen) with 0.15% w/v bovine serum albumin (Sigma-Aldrich) for washing. The PBMCs were washed twice for 7 minutes at 1500 rpm. Peripheral blood lymphocytes (PBLs) were isolated via panning PBMCs onto a culture flask for 30 minutes at 37° C., allowing monocytes to adhere. PBLs were then counted and re-suspended at 1×10⁶ cells/ml in M199 (Gibco Invitrogen) with 0.15% w/v bovine serum albumin (Sigma-Aldrich) for the transmigration assays.

### Cell Culture

Human dermal blood endothelial cells (HDBECS, Promocell) were cultured in a low serum 2% (Endothelial cell growth medium; (PromoCell) with 35 μg/ml gentamycin (Gibco). The cells were frozen down at passage 4 in CryoSFM (PromoCell) and stored in liquid nitrogen.

### In Vitro Transmigration Assay

HDBECS defrosted at 37° C. in a 25 cm² cell culture flask in 10 ml of media were dissociated using EDTA (Sigma-Aldrich) and Trypsin (Gibco) and seeded on 10 wells in a 12-well tissue culture plates (Falcon) and left to adhere at 37° C. After 24 hours, confluent endothelial layers were cytokine stimulated using 100 U/ml TNF-α (Sigma-Aldrich) and 10 ng/ml IFN-γ (Peprotech) for 24 hours at 37° C. The peptide treatment of the 1×10⁶ cells/ml PBLs consisted of adding the peptides immediately before use. Before the addition of the PBLs, the endothelial layers were washed with M199 BSA (0.15% w/v), removing any surplus cytokine. 1×10⁶ PBLs were added to each well and incubated at 37° C., allowing PBLs to adhere to migrate across the endothelium. To remove any non-adherent cells, the PBLs were washed twice with M199 BSA (0.15% w/v) after 6 minutes. Cells were then fixed with 2% glutaraldehyde for 10 minutes and then imaged using phase-contrast microscopy via an inverted bright-field microscope at 20× magnification.

### Analysis

Imaging of the PBL migration assay was carried out using ImagePro 7 software (Media Cybernetics). Surface adherent cells were categorised as being phase-bright, and transmigrated cells were categorised as phase-dark with altered morphology, appearing as being under the endothelial layer. Images were taken at 5 different fields of each well, and an average of counted adherent and migrated PBLs was calculated.

### Calculation of IC₅₀

The same methods described for the in vitro transmigration assay and analysis were used, and PBL transmigration was calculated as a percentage of adherent cells. Peptides were added to the PBLs at concentrations ranging from 0.0001-500 ng/ml on the cytokine stimulated HDBECs and the resulting PBL transmigration (as a percentage of adherent cells) was plotted as a function of log₁₀ of the peptide concentration. The IC₅₀ value may be calculated from 10^{x-coordinate}, where the x-coordinate is the value of x (log₁₀ of the peptide concentration) that gives rise to the half-maximum inhibition response, i.e. that gives rise to the y-coordinate (PBL transmigration as a percentage of adherent cells) value that is equal to smallest value of y subtracted from the largest value of y.

### T_{1/2} Assays

T_{1/2} in Plasma The method used to determine T_{1/2} values of the peptides in plasma is as follows:
1) Add solvent, typically 120-200 μl (10% trichloroacetic acid (TCA), acetonitrile or methanol) to tubes comprising plasma. Store at −20° C. or on ice.
2) Thaw the plasma and spin in a centrifuge at 1500×g for 10 minutes at room temperature.
3) Prepare stock solution of the peptide at 10 mM in dimethyl sulfoxide (DMSO) or phosphate buffered saline (PBS) and dilute solutions by adding 3 μL of 10 mM stock to 297 μL DMSO or PBS (100 UM stock for a final 1 μM concentration).
4) Add 550 μL plasma to an incubation tube and separately add 5 μL of peptide stock solution to an incubation tube. Warm the incubation tubes (5 minutes, 37° C., with shaking).
5) After warming, add 495 μL of warmed plasma to warmed peptide (time=0). Mix well and shake the tube containing the mixed sample (37° C., 1500×g).
6) At 2, 5, 15, 30, 45 and 60 minutes after contacting the peptide and plasma, collect 40 μL of the sample into pre-prepared quench tubes.
7) Analyse the sample at each time point by mass spectrometry. Take all measurements in duplicate.

### T_{1/2} in Blood

The method used to determine T_{1/2} values of the peptides in plasma is as follows:
1) Add solvent, typically 120-200 μl (10% TCA, acetonitrile, methanol, or a mixture of acetonitrile/methanol) to tubes comprising blood. Store at −20° C. or on ice.
2) Thaw the blood and spin in a centrifuge at 1500×g for 10 minutes at room temperature.
3) Prepare stock solution of the peptide at 10 mM in DMSO or PBS and dilute solutions by adding 3 μL of 10 mM stock to 297 μL DMSO or PBS (100 μM stock for a final 1 μM concentration).
4) Add 550 μL blood to an incubation tube and separately add 4 μL of peptide stock solution to an incubation tube. Warm the incubation tubes (5 minutes, 37° C., with shaking).
5) After warming, add 396 μL of warmed blood to warmed peptide (time=0). Mix well and shake the tube containing the mixed sample (37° C., 1400×g).
6) At 2, 5, 15, 30, 45 and 60 minutes after contacting the peptide and blood, collect 40 μL of the sample into pre-prepared quench tubes.
7) Analyse the sample at each time point by mass spectrometry. Take all measurements in duplicate.

The samples were analysed and the T_{1/2} values were calculated by XenoGesis. The T_{1/2} values correspond to the time taken for the concentration of the peptide in the blood or plasma to be reduced by 50%. They are typically calculated from plots of concentration of the peptide detected in the blood or plasma as a function of time. The peptide in each quench tube may be detected quantitatively using mass spectrometry (see, for example, Chung T D Y, Terry D B, Smith L H. In Vitro and In Vivo Assessment of ADME and PK Properties During Lead Selection and Lead Optimization—Guidelines, Benchmarks and Rules of Thumb. 2015 Sep. 9. In: Sittampalam G S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK326710/.)

### Initial Results

To identify whether the core functional motif of PEPITEM lies within the first 7 amino acids, various derivatives of PEPITEM containing either all of the first 7 amino acids, or different regions of the first 7 amino acids were synthesised. Each peptide contained a tripeptide of structure SVT or QGA. Transmigration assays using the peptides of Table 4 were carried out on seeded HBECS stimulated with 100 U/ml of TNF-α and 10 ng/ml of IFN-γ for 24 hours. The peptides were added at concentration of 20 ng/ml and run alongside untreated PBL (PBL0) and scrambled protein to analyse their effects on adhesion and transmigration.

TABLE 4

| Peptide ID | Amino acid Sequence |
|---|---|
| PEPITEM | SVTEQGAELSNEER |
| (SEQ ID NO. 1) | |
| Scrambled PEPITEM | TAVLENSREGESQE |
| (SEQ ID NO. 41) | |
| SEQ ID NO. 2 | SVT |
| SEQ ID NO. 34 | SVTE |
| SEQ ID NO. 35 | SVTEQ |
| SEQ ID NO. 36 | SVTEQG |
| SEQ ID NO. 37 | SVTEQGA |
| SEQ ID NO. 38 | EQGA |
| SEQ ID NO. 39 | VTEQGA |
| SEQ ID NO. 42 | AVT |
| SEQ ID NO. 43 | SAT |
| SEQ ID NO. 44 | SVA |
| SEQ ID NO. 45 | AGA |
| SEQ ID NO. 46 | Ac-QAA |
| SEQ ID NO. 40 | SVTQAA |

The peptides did not have a statistically significant effect on the adhesion of PBLs, however all peptides caused a significant decrease in the transmigration of PBLs (FIG. 2). Each peptide exhibited a statistically significant decrease when compared with the transmigration results for PBL0, thus all were efficacious in inhibiting trans-endothelial migration of PBLs.

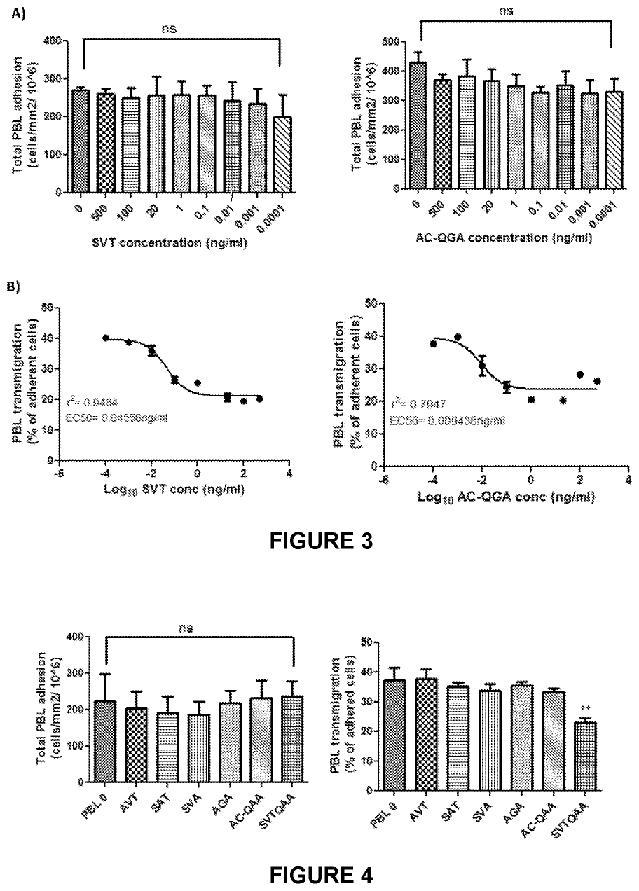

Efficacy of peptides containing SVT and QGA sequences prompted analysis between the two to identify if one is more effective than the other (FIG. 3). No significant difference between the two tripeptides was seen, with P>0.05. A dose response for each of the tripeptides was carried out. The peptides were added at concentrations ranging from 0.0001-500 ng/ml on cytokine stimulated HDBECs. Neither the SVT tripeptide nor the QGA tripeptide exhibited any significant effect on PBL adhesion when compared to unstimulated PBL. Surprisingly, the SVT tripeptide possessed a very similar dose response to that of PEPITEM, with an IC₅₀ of 0.04558 ng/ml, whereas the QGA tripeptide inhibited the trans-endothelial migration of PBLs with an IC₅₀ of 0.009438 ng/ml (FIG. 3). The lower IC₅₀ value of QGA indicates that peptides comprising this amino acid sequence may be more potent and potentially more efficacious than peptides comprising the SVT sequence.

Results with Alanine-Substituted Peptides

Transmigration assays with alanine-substituted peptides were carried out in order to assess the effect of each amino acid in the SVT and QGA tripeptide sequences on the ability of the peptide to inhibit T-cell migration. Single alanine substitutions at different residues of peptides comprising sequences SVT, QGA or SVTQGA were carried out. The use of alanine prevents interaction with the overall structure and function of the peptide as alanine is a charge-neutral amino acid.

The alanine substituted peptides of the sequences shown in Table 4 were added to HDBECS stimulated with 100 U/ml of TNF-α and 10 ng/ml of IFN-γ for 24 hours. The effects of alanine substitution on both adherence and transmigration were analysed in FIG. 4. As expected, none of the alanine substituted peptides inhibited the adhesion of PBLS. All but the peptide of sequence SVTQAA lacked any statistically significant effect on trans-endothelial migration. The peptide of sequence SVTQAA caused a significant (p<0.01) fall in PBL transmigration when compared to PBL0 (23% when compared to 37%).

These data confirm the importance of each amino acid in the SVT and QGA tripeptide sequences in enabling the peptide to inhibit T-cell migration.

Results with Tripeptide Derivatives

In this work, over 30 tripeptide derivatives of PEPITEM were designed, made and tested for their ability to inhibit T cell migration and their stability in plasma and blood. The results are shown in Tables 5 and 6.

TABLE 5

| | | | | | | T_{1/2} (min) | |
|---|---|---|---|---|---|---|---|
| | | | % Inhibition | | IC₅₀ | mouse | human |
| Ref. | Structure | MW | (0.3 nM) | (3 nM) | (nM) | plasma | plasma |
| SEQ ID NO. 1 | SVTEQGAE | 1,548.58 | | 41.90 | 0.0200 | >180 | >180 |
| | LSNEER | | | | | | |
| SEQ ID NO. 2 | H-SVT | 305.33 | 42.20 | | 0.1500 | 20 | |
| SEQ ID NO. 3 | Ac-SVT | 347.37 | | 36.80 | | | |
| SEQ ID NO. 4 | H-SVT-NH₂ | 304.35 | | 38.50 | 0.0100 | >180 | >180 |
| SEQ ID NO. 5 | Ac-SVT-NH₂ | 346.38 | | 42.50 | | 119 | |
| SEQ ID NO. 6 | H-SVT-NH- | 332.40 | 46.00 | | 0.0170 | >180 | >180 |
| | Ethyl | | | | | | |
| SEQ ID NO. 7 | H-S*VT-NH₂ | 304.35 | 9.50 | | | | |
| SEQ ID NO. 8 | H-TVT-NH₂ | 318.37 | 45.47 | 48.19 | | 17 | |
| SEQ ID NO. 9 | H-SVS-NH₂ | 290.32 | 42.51 | 38.10 | | 103 | |
| SEQ ID NO. 10 | H-S*V*T*- | 304.35 | 24.76 | 39.51 | | | |
| | NH₂ | | | | | | |
| SEQ ID NO. 11 | H-T*V*S*- | 304.35 | 56.89 | 58.74 | 0.0240 | >180 | |
| | NH₂ | | | | | | |
| SEQ ID NO. 12 | H- | 318.37 | 34.72 | 62.27 | | | |
| | S(OMe)VT- | | | | | | |
| | NH₂ | | | | | | |
| SEQ ID NO. 13 | H-SVT-NH- | 388.51 | 23.56 | 12.83 | | | |
| | Hexyl | | | | | | |
| SEQ ID NO. 14 | H-SIT-NH₂ | 318.37 | 3.40 | 28.23 | | | |
| SEQ ID NO. 15 | H-SLT-NH₂ | 318.37 | 46.00 | 20.75 | | 7 | |
| SEQ ID NO. 16 | H-SFT-NH₂ | 352.39 | 39.78 | 33.33 | | | |
| SEQ ID NO. 17 | H-SWT-NH₂ | 391.43 | 46.69 | 74.47 | 0.0236 | 74 | |
| SEQ ID NO. 18 | H-SYT-NH₂ | 368.39 | 39.18 | 42.83 | | 17 | |

TABLE 6

| | | | | | | T1/2 (min) | | |
|---|---|---|---|---|---|---|---|---|
| | | | % Inhibition | | IC₅₀ | mouse | human | mouse |
| Ref. | Structure | MW | (0.3 nM) | (3 nM) | (nM) | plasma | plasma | blood |
| SEQ ID NO. 1 | SVTEQGAE | 1,548.58 | | 41.90 | 0.0200 | >180 | >180 | 41 |
| | LSNEER | | | | | | | |
| SEQ ID NO. 19 | Ac-QGA | 316.31 | | | 0.0300 | | | |
| SEQ ID NO. 20 | Ac-QGA-NH₂ | 315.33 | | 28.60 | 0.1000 | 49 | >180 | |
| SEQ ID NO. 21 | PEGA | 257.25 | | 41.20 | 0.1200 | >180 | >180 | |
| SEQ ID NO. 22 | PEGA-NH- | 284.32 | 35.40 | | | >180 | | |
| | Ethyl | | | | | | | |
| SEQ ID NO. 23 | PEGA-NH₂ | 256.26 | 54.50 | | 0.0032 | >180 | | |
| SEQ ID NO. 24 | Ac-QGA-N_{H}- | 343.38 | 50.00 | | | | | |
| | Ethyl | | | | | | | |
| SEQ ID NO. 25 | Ac-QGAib | 330.34 | 32.00 | | | | | |
| SEQ ID NO. 26 | Ac-Q*GA*- | 315.33 | 33.50 | | 0.0180 | >180 | | |
| | NH₂ | | | | | | | |
| SEQ ID NO. 27 | Ac-NGA | 301.30 | 32.78 | 44.52 | | | | |
| SEQ ID NO. 28 | pE*GA*-NH₂ | 256.26 | 46.18 | 50.93 | | | | 70 |
| SEQ ID NO. 29 | PEGA-NHMe | 270.29 | 40.89 | 43.35 | | | | 97 |
| SEQ ID NO. 30 | PEGA- | 284.32 | 44.70 | 42.65 | | | | >180 |
| | N(Me)₂ | | | | | | | |
| SEQ ID NO. 31 | pEGV-NH₂ | 284.32 | 49.94 | 50.27 | | | | >180 |
| SEQ ID NO. 32 | pEGL-NH₂ | 298.34 | 25.34 | 58.16 | | | | |
| SEQ ID NO. 33 | pEGF-NH₂ | 332.36 | 49.15 | 42.84 | | | | 146 |

We have now shown that certain tripeptides of the invention are at least as potent as PEPITEM (i.e. have an IC₅₀≥0.02 nm), and are in some cases more potent at inhibiting the transmigration of T cells, such as SEQ ID NOs 4, 6, 23 and 26. Certain tripeptides are at least as effective in inhibiting PBL transmigration (as a percentage of adherent cells) as PEPITEM (i.e. have a % Inhibition at 0.3 nm and/or 3 nm that is ≥41.90), and are in some cases more effective, such as SEQ ID NOs 2, 5, 6, 8, 9, 11, 12, 15, 17, 18, 23, 24, and 27 to 33. Certain tripeptides are at least as stable in mouse plasma, human plasma, and or mouse blood as PEPITEM (i.e. have a T_{1/2} (min) in mouse plasma of ≥180, in human plasma of ≥180, and in mouse blood of ≥41), for example SEQ ID NOs 4, 6, 11, 20, 21 to 23 and 26. In some cases, the tripeptides of the invention are more stable in mouse blood, such as SEQ ID NOs 28 to 31 and 33.

Treatment of Osteoblasts with Peptides

### In Vitro Assays

### Human Osteoblasts

Human osteoarthritis subchondral joint tissue was obtained at the time of total knee and hip joint replacement operations from the Royal Orthopaedic Hospital (Birmingham). To isolate primary osteoblasts, cartilage was removed from the femoral condyles and tibial plateaus and cut into 2 mm² pieces. Samples were kept in media and cleaned of fat. Media contains DMEM (Sigma, D6546), FCS (10%), GPS (1%), Non-essential amino acid (1%; Sigma, M7145), β10 glycerophosphate (2 mM; Sigma, G9422) and L-Ascorbic acid (50 μg/ml; Sigma, A5960). Bone chips were placed in T₇₅ flasks in 10 mL of media, replacing media every 2-3 days. Main outgrowth occurs between 10-14 days, after which the chips were placed in new flasks. At 90% confluence, the cells were trypsinised and used or split for further culture.

### Murine Primary Calvarial Osteoblasts

Day 3-5 WT mouse pups were culled via decapitation and heads were washed briefly in 70% ethanol and kept hydrated in αMEM media supplemented with 2 mM L-Glutamine, 100 U/ml Penicillin and 100 μg/ml Streptomycin. Skin was removed from around calvaria using forceps, and the calvaria was dissected out and cleaned of any contaminating tissue. Calvaria were added to αMEM media supplemented with 1 mg/ml of collagenase d (Roche, Basel, Switzerland, COLLD-RO). Tubes were then agitated at 37° C. for 10 minutes and the media discarded. αMEM media supplemented with 1 mg/ml of collagenase d was again added to calvaria and shaken at 37° C. for 30 minutes. Media (containing cells) was then removed and kept on ice Media (containing cells) was then removed and kept on ice. The calvaria were then again shaken at 37° C. for 10 minutes in 1.5 ml of αMEM media supplemented with 5 μM EDTA (Sigma-Aldrich, E7889) and the cell containing media transferred to the sample on ice, pooling the cells. Finally, the calvaria was incubated with 1 mg/ml collagenase d in αMEM and agitated at 37° C. for 30 minutes, before cell containing media was pooled with the cells on ice collected from the 2 previous wash steps. Cells on ice were then spun down at 300 g for 4 minutes, resuspended in 1 ml of Basal media and counted using a haemocytometer. Cells were then diluted to 1×10⁶ cells/ml and split among culture flasks with additional Basal media. After 24 hours, media was changed to remove non-adhesive and contaminated cells. Primary calvarial osteoblasts were then utilised at P1, or frozen down for analysis later.

### Effect of Peptide on Osteoblast Cells

Osteoblasts (8×10³ cells/well) were cultured in mineralisation differentiation media in the presence or absence of peptide (10 ng/ml) for 4 days. Mineralisation was assessed by quantifying the level of alkaline phosphate (ALP) activity (absorbance at 405 nm) per condition normalized to the absorbance generated by the untreated control (control on the graph).

### Alkaline Phosphatase Assay

To measure ALP activity, cells were washed in PBS before being lysed in 100 μl of RIPA buffer (Thermo Fisher, R⁰²⁷⁸) for 15 minutes on ice. Cells were harvested using a cell scraper, vortexed and centrifuged at 13000 g for 10 minutes. From the cell lysates, 10 μL was added to 90 μL of alkaline phosphatase yellow (pNPP) liquid substrate for ELISA (Sigma-Aldrich, P7998) in a 96 well plate. The plate was wrapped in foil and left to run for 45 minutes at 37° C. in an incubated shaker (SciQuip; Shropshire, UK) before being quantified using a Synergy HT plate reader (BioTek) with absorbance set at 405 nm.

### Monoarthritis Model

Mice were injected (sub-cutaneously) on one occasion with 100 μl antigen (bovine serum albumin; BSA) containing Freund's complete adjuvant (CFA) into a maximum of two sites in the lower dorsal area. At day 21, the mice were injected (10 μl, intra-articular) with the same antigen alone, in the absence of CFA, in one knee joint and PBS in the contralateral knee joint as a non-inflamed control. Animals received immune modulating agents (PEPITEM; SVT; QGA; or PBS as a control) by intraperitoneal injection at time 0, 24 h, 48 h and 72 h. Joint swelling as a measure of inflammation was conducted using calibrated calipers.

### Results

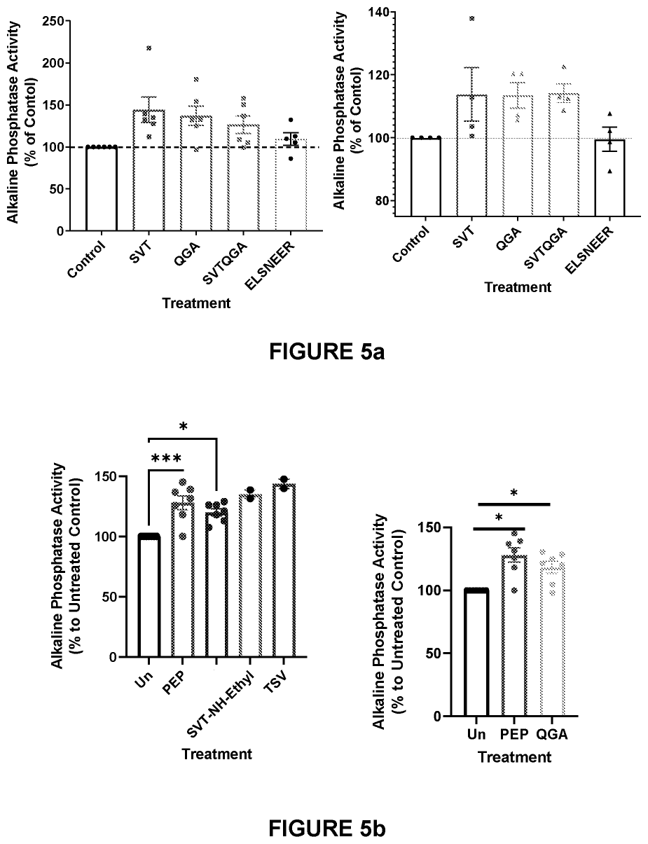

The data presented in FIG. 5a and FIG. 5b shows that the peptides disclosed herein stimulate the production of bone mineral by both primary murine and human osteoblasts when the cells are cultured in isolation in vitro. To obtain the data in FIG. 5a, primary murine and human osteoblasts were allowed to mineralise for 4 days in the presence or absence of peptide. Mineralisation was assessed by quantification of ALP activity in murine or human osteoblasts. The 3-7mers disclosed herein significantly increased murine and human primary osteoblast mineralization with respect to the untreated cells. We note that the osteoblasts treated with the 7mer of sequence ELSNEER (SEQ ID NO. 47) did not exhibit a statistically significant change in osteoblast activity. This peptide sequence does not comprise formula (I) or formula (II), and this result further supports the importance of formula (I) and formula (II) in peptide efficacy.

The data presented in FIG. 5b was obtained by allowing primary murine osteoblast cells to mature and mineralise over 8 days in the presence or absence of PEPITEM (PEP), SVT, SVT-NH-ethyl or TSV. Mineralisation, as a measure of bone formation, was assessed by quantification of Alkaline Phosphatase Activity in the osteoblasts. PEPITEM and the tri-peptides tested significantly increased murine primary osteoblast maturation mineralisation. *=p<0.05 and ***=p<0.001 by Dunn test compared to untreated cells.

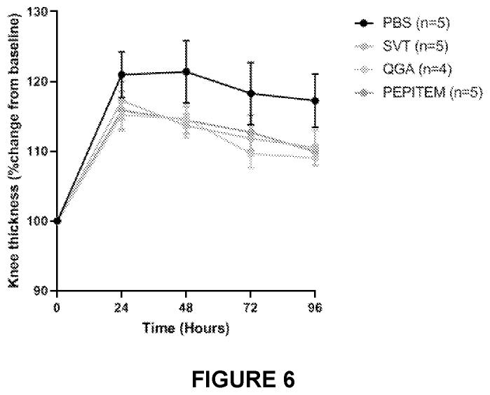

The data presented in FIG. 6 shows that the peptides disclosed herein reduce knee swelling in a model of antigen induced monoarthritis. Joint swelling is an indirect measurement of inflammation within 24 hours of intra-articular administration of the antigen (m-BSA). PEPITEM, SVT and QGA were equipotent in limiting joint swelling.

### Effect of Peptides on Leukocyte Cell Migration

### Peritonitis Mouse Model

To measure the effect of the peptides disclosed herein on the number of leukocytes and macrophages in the peritoneum of mice, 100 μg of Zymosan and 100 μg of the peptide (0.646 mM) were administered by intraperitoneal injection into mice. 4 hours after administration, the mice were sacrificed and a peritoneal lavage was conducted with 5 mL of cold PBS and 0.5 mM EDTA. Flow cytometry was used to assess the population of various leukocytes and cell counts was performed. Fluids were stored −80° C. for mediator analysis.

### Results

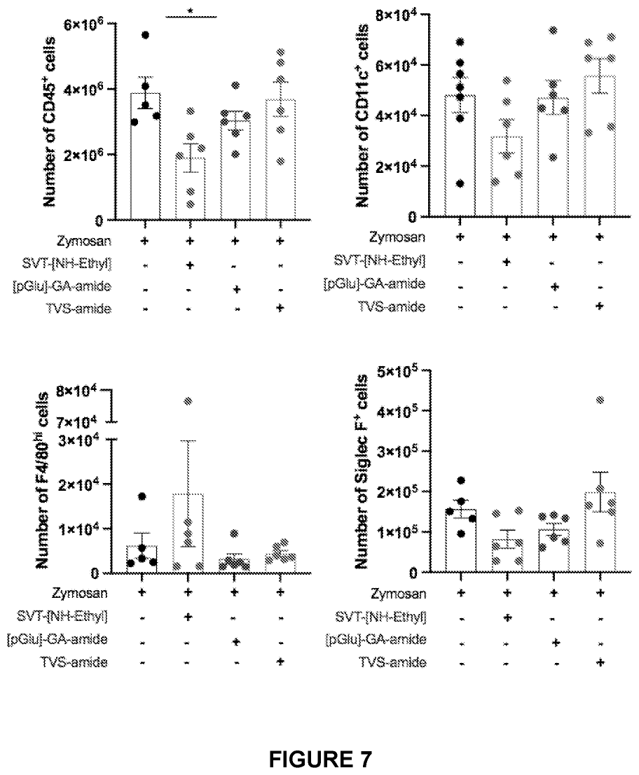

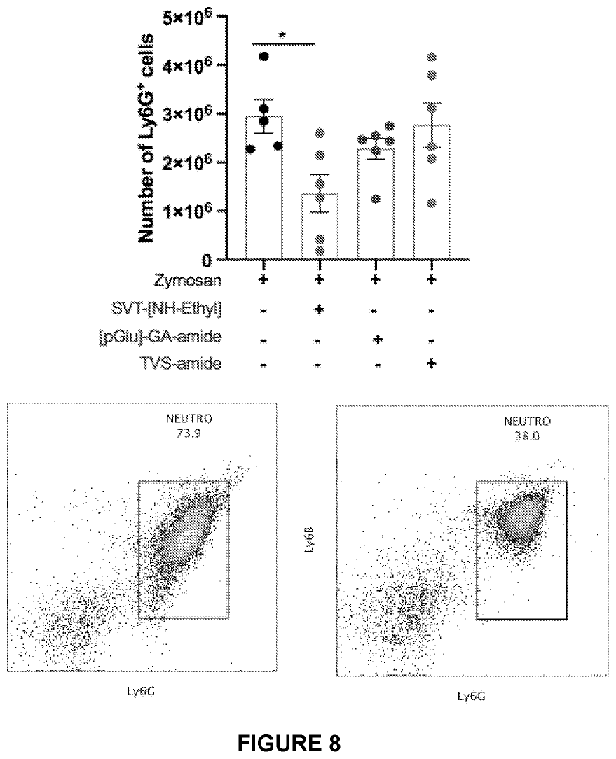

The data presented in FIG. 7 shows that the peptides disclosed herein reduce the number of leukocytes counted in the peritoneum of mice after 4 hours of treatment with Zymosan and the peptide. A lower cell number corresponds to reduced trafficking of leukocytes and a reduced inflammatory response. The data presented in FIG. 8 shows that the peptides disclosed herein reduce the number of neutrophils counted in the peritoneum of mice after 4 hours of treatment with Zysoman and the peptide.

### Effects of Peptides on Gouty Arthritis

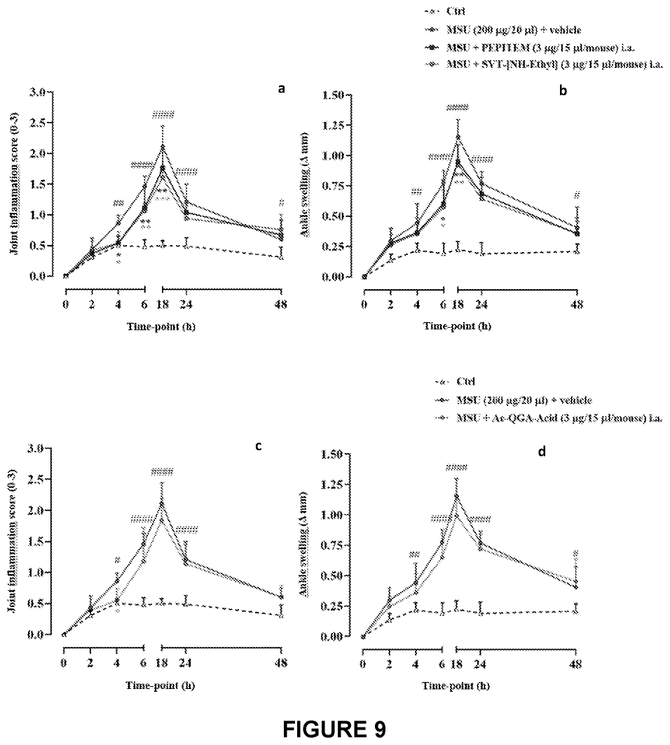

Mice were treated with PEPITEM (3 μg/mouse i.a.), SVT-[NH-Ethyl] (3 μg/mouse i.a.) or Ac-QGA-Acid (3 μg/mouse i.a.) 30 minutes after intra-articular stimulation with MSU crystals (200 μg/20 μl) in the right knee joints. Joint inflammation score (0-3 in increments of 0.25) was evaluated at 2, 4, 6, 18, 24, and 48 h after MSU injection (see FIG. 9(a) and FIG. 9(c)). Joint inflammation edema was evaluated at 2, 4, 6, 18, 24 and 48 h after the stimulus with MSU (see FIG. 9(b) and FIG. 9(d)). Data are expressed as joint inflammation score (C), Δ increase of knee joints mm (D) and presented as means±S.D. of n=5 mice per group. Statistical analysis was conducted by two-way ANOVA followed by Dunnett's for multiple comparisons. ^{#}P≤0.05, ^{##}p<0.01, ^{####}P≤0.0001 vs Ctrl group; *P≤0.05, **P≤0.01, ***P≤0.001 vs MSU group.

The data presented in FIG. 9 shows that the peptides disclosed herein reduced inflammation associated with gouty arthritis (gout). In some instances, peptides of formula (I) reduced inflammation to a greater degree than PEPITEM.

### Effects of Peptides on Peritonitis

Wild type mice were injected by the intraperitoneal route with 0.1 mg of sterile zymosan, stimulating peritonitis. After 48 hours (for the T cell and B cell tests) or 4 hours (for the neutrophil tests), the inflammatory leukocytic infiltrate was collected by peritoneal lavage. Leukocytes were washed and labelled with sub-set specific antibodies conjugated with fluorescent markers. The number of each sub-set was analysed by flow cytometry.

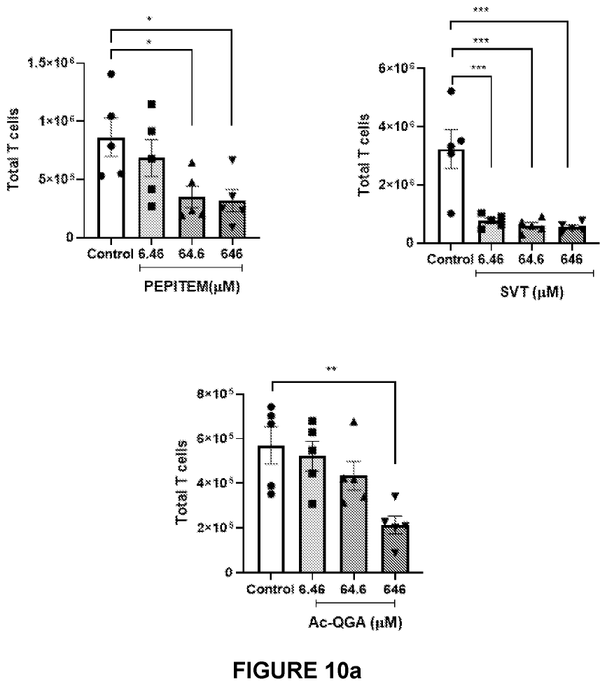

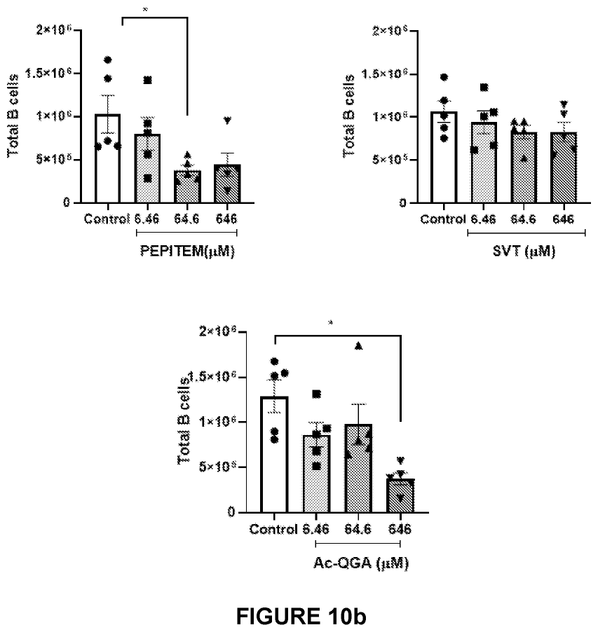

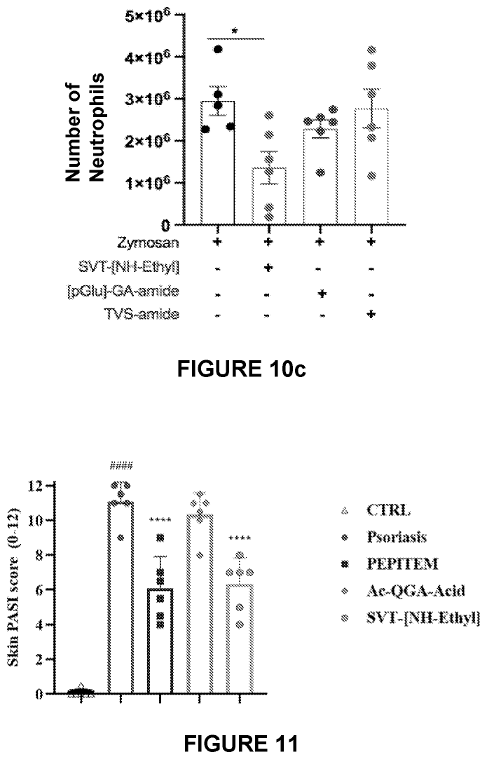

The data presented in FIG. 10a show that the peptides disclosed herein reduced the number of T cells in the inflammatory leukocytic infiltrate of mice with peritonitis, supporting a reduction in the inflammatory immune response. The data presented in FIG. 10b show that certain peptides disclosed herein (the QGA tripeptide in particular) reduce the number of B cells in the inflammatory leukocytic infiltrate of mice with peritonitis, supporting a reduction in the inflammatory immune response. The data presented in FIG. 10c show that certain peptides disclosed herein (the SVT tripeptide in particular) reduce the number of neutrophils in the inflammatory leukocytic infiltrate of mice with peritonitis, also supporting a reduction in the inflammatory immune response.

### Effects of Peptides on Psoriasis

The necks of experimental mice were shaved prior to the assay. 62.5 mg (5% imiquimod)±PEPITEM or tripeptides were applied to the skin daily for 7 days. Disease severity was assessed by integrated PASI score (0-12) comprising: levels of erythema (0-4)+skin scaling (0-4)+skin thickness (0-4).

The data presented in FIG. 11 show that particular peptides disclosed herein (such as SVT tri-peptides) reduce the severity of imiquimod-induced psoriasis at least to the same extent as PEPITEM.

Effects of Peptides on Cytokine Release from Macrophages

Murine macrophage cell line J774 was established in culture. Cells were treated with PEPITEM, tripeptides, or peptide-mimetics for 15 minutes or had no treatment prior to the addition of LPS (a phlogistic stimulus). Supernatants were collected at 24 h. IL-6 and TNF-α concentrations in the supernatants were measured using ELISA. Data are from 3 experiments, ####=p<0.0001 vs control group; *=p<0.05: **=P≤0.01 vs LPS, by ANOVA.

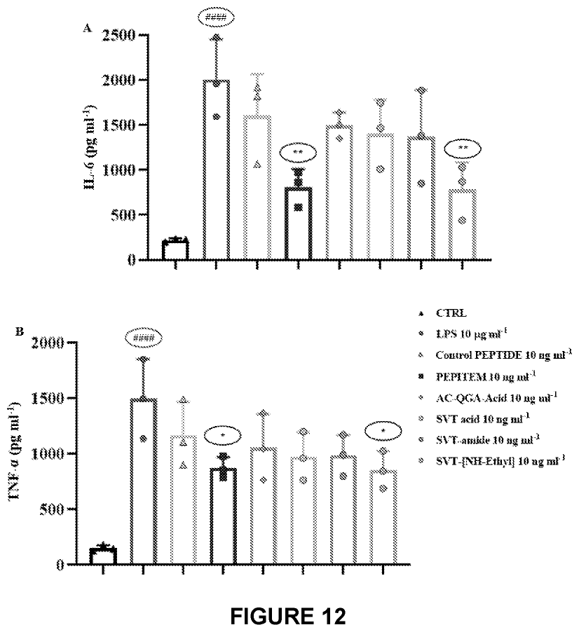

The data presented in FIG. 12 show that particular peptides disclosed herein (such as SVT-[NH-Ethyl] tri-peptides) reduce the concentration of IL-6 (FIG. 12A) and TNF-α (FIG. 12B) released from macrophages at least to the same extent as PEPITEM, supporting the ability of the peptides to reduce the inflammatory immune response.

Effects of Peptides on Cytokine Release from Fibroblasts

NIH3T3 mouse embryonic fibroblast cells were established in culture. Cells were treated with PEPITEM, tripeptides or peptide-mimetics for 15 minutes or had no treatment prior to the addition of LPS. Supernatants were collected at 24 h. IL-6 and TNF-α concentrations in the supernatants were measured using ELISA. Data are from 3 experiments, ###=p<0.001, ##=p<0.01 vs control group; *=p<0.05: **=P≤0.01 vs LPS, by ANOVA.

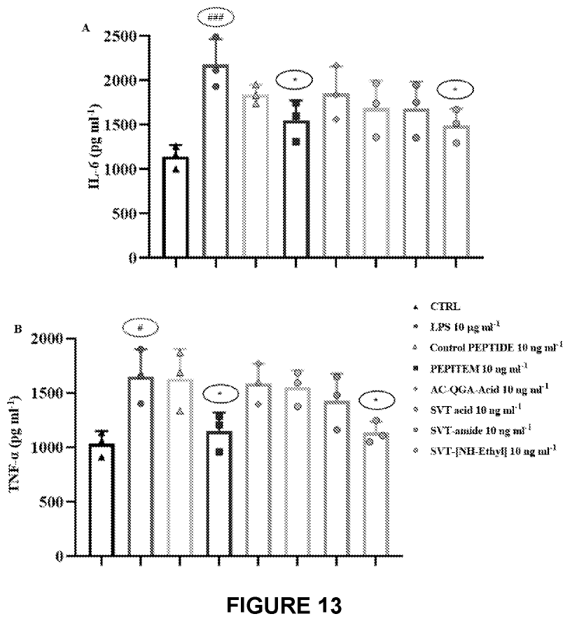

The data presented in FIG. 13 show that particular peptides disclosed herein (such as SVT-[NH-Ethyl] tri-peptides) reduce the concentration of IL-6 (FIG. 13A) and TNF-α (FIG. 13B) released from macrophages at least to the same extent as PEPITEM, supporting the ability of the peptides to reduce the inflammatory immune response.

### Effects of Peptides on Keratinocyte Proliferation

HaCaT human epidermal keratinocytes were established in culture in 96 well plates. Cells were treated with PEPITEM, tripeptides or peptide-mimetics for 15 minutes or had no treatment prior to the addition of M5 cytokines (IL-17A, IL-22, IL-1α, OSM and TNFα). After 72 hours, MTT substrate was added to the wells and incubated for 3 hours. Colourimetric intensity as a measure of proliferation was assayed using a plate reader.

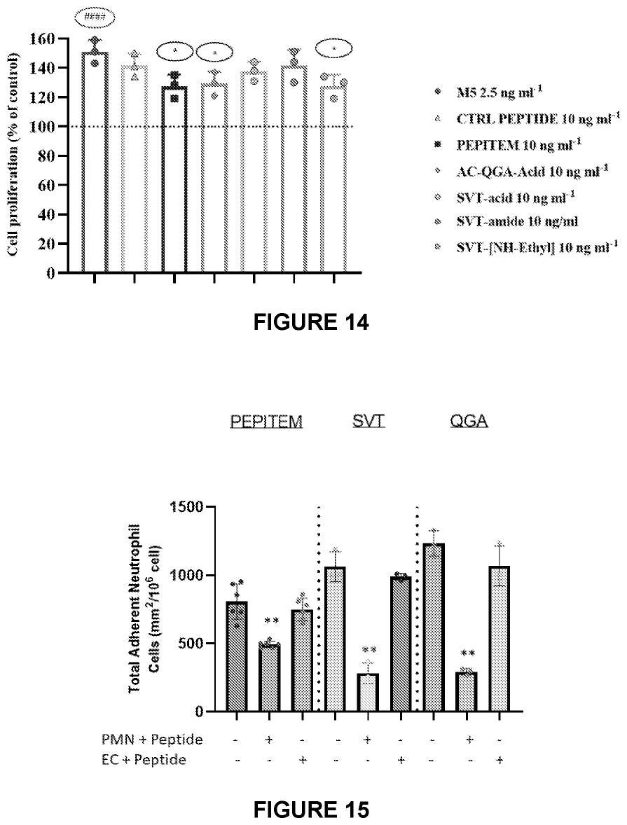

The data presented in FIG. 14 show that particular peptides disclosed herein (such as Ac-QGA-Acid or SVT-[NH-Ethyl] tri-peptides) reduce cell proliferation of keratinocytes stimulated to proliferate by M5 cytokines at least to the same extent as PEPITEM, supporting the ability of the peptides to reduce the inflammatory immune response.

### Effects of Peptides on Neutrophil Adhesion to TNF-α-Stimulated Endothelial Cells

Cultured endothelial cells (ECs) were stimulated with 100 U of TNF-α for 4 hours. Neutrophils were isolated from freshly drawn whole blood. Neutrophils or ECs were pre-treated with PEPITEM, SVT, or QGA for 15 minutes prior to adhesion assay or were untreated. Neutrophils were added to wells containing stimulated ECs and incubated for 15 minutes. Wells were fixed and none-adherent neutrophils washed out. Adhesion was assessed by phase contrast microscopy. Data are form at least 3 independent experiments. **=p<0.01 by ANOVA.

The data presented in FIG. 15 show that neutrophil adhesion to endothelial cells is reduced when the neutrophils are pre-treated with the peptides disclosed herein. Particular tripeptides (such as SVT and QGA) were able to reduce neutrophil adhesion to a greater extent than PEPITEM.

Penetration of Peptides into the Eye when Administered Topically

Pig eyes were treated with either PEPITEM or one of TVS (reverse sequence of SVT comprising amino acids in D-isomeric forms), SVT, EGA and QGA, derived from the PEPITEM sequence. The eyes were then incubated in a dark humified chamber. The eyes were dissected, and the vitreous humour and retina were harvested. The samples were sonicated to break up the tissue and purified using a C18 column. Once purified, the samples were digested and analysed by mass spectrometry.

The full PEPITEM sequence was found in the retina sample but not in the vitreous sample. For the SVT (Vitreous humour & Retina), TVS (Retina only), EGA (Retina only) and QGA (Retina only), mass spectra show m/z readings very similar to the molecular weights of the small peptides and/or show possible aggregation of the trimers. For instance, SVT has a molecular weight of 305 but in the mass spectrum a peak at 306 can be found as well as a peak at 611, which shows possible aggregation. These slightly higher m/z values are in keeping with previous data obtained when the trimers alone were tested for reference.

1. A peptide of 3-7 amino acid residues, wherein the peptide is configured for administration as a medicament and comprises formula (I) and/or formula (II); wherein formula (I) is: 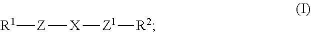
wherein Z and Z¹ are each independently an amino acid selected from serine and threonine; X is an amino acid selected from valine, leucine, phenylalanine, tryptophan and tyrosine; R¹ is hydrogen (H), COR³ or a bond to another amino acid; R² is OH, N(R⁴)₂ or a bond to another amino acid; and each R³ and R⁴ is independently selected from H and a C₁-C₆ alkyl group; and
formula (II) is: 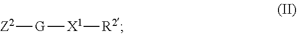
wherein Z² is selected from N, Q, Ac—N, Ac-Q or pyroglutamic acid, wherein Q represents glutamine and N represents asparagine; G represents glycine; X¹ is an amino acid selected from alanine, valine, leucine, phenylalanine and 2-amino-2-methylpropanoic acid; R^{2′} is OH, N(R^{4′})₂ or a bond to another amino acid; and each R⁴ is independently selected from H and a C₁-C₆ alkyl group; and
wherein the peptide is not of the sequence CSVTCG.

2. The peptide of claim 1, wherein the peptide is represented by any one of formulae (I), (II), (Va) to (Vd), (VIa to VIf), (VIIa) to (VIIh), (VIIIa) to (VIIIh), (IIIa) to (IIIf), (IVa) and (IVb): 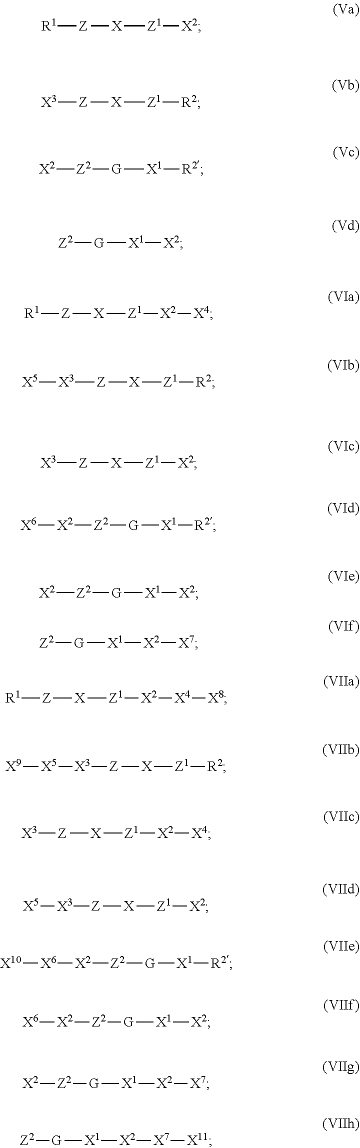 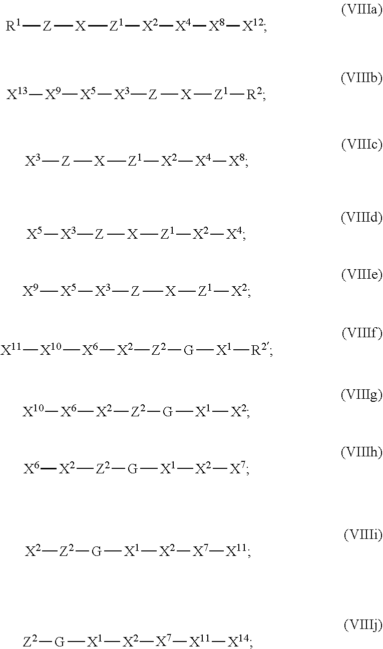 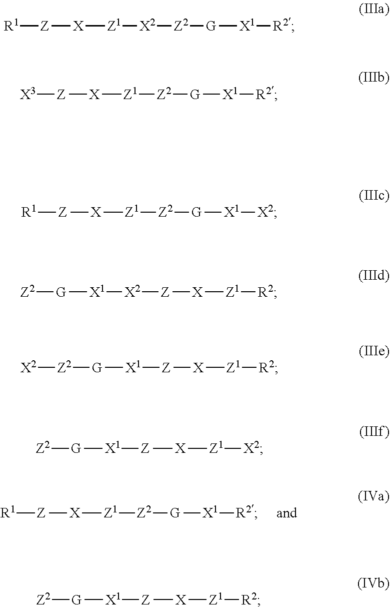
wherein X² to X¹⁴ are each an amino acid; R¹ is H or COR³; R^{2′} is OH or N(R^{4′})₂; R² is OH or N(R⁴)₂; Z² of formulae (Vc), (VId), (VIe), (VIIe) to (VIIg), (VIIIf) to (VIIIi), (IIIa) to (IIIc), (IIIe) and (IVa) is N or Q; and Z² of formulae (II), (Vd), (VIf), (VIIh), (VIIIj), (IIId), (IIIf) and (IVb) is Ac—N, Ac-Q or pyroglutamic acid.

3. The peptide of claim 2, wherein the peptide is represented by any one of formulae (I), (II), (Va) to (Vd), (VIa), (VIc) to VIf), (VIIc), (VIIe) to (VIIh), (VIIIa) to (VIIIh), (IIIa) to (IIIf), (IVa) and (IVb).

4. The peptide of claim 2, wherein X² is glutamic acid; X⁴ is glutamine; X⁶ is threonine; X⁷ is leucine; X⁸ is glycine; X¹⁰ is valine; X¹¹ is serine; X¹² is alanine; and X¹⁴ is asparagine.

5. The peptide of claim 2, wherein Z is serine and X is any one selected from valine, tryptophan and tyrosine.

6. The peptide of claim 2, wherein Z—X—Z¹ is selected from SVT; SLT; SFT; SWT; SYT; TVT; SVS; and TVS.

7. The peptide of claim 2, wherein X¹ is an amino acid selected from alanine, valine, leucine and phenylalanine.

8. The peptide of claim 2, wherein Z²-G-X¹ is any one selected from QGA; NGA; Ac-QGA; pEGA; Ac-QGAib; Ac-NGA; pEGV; pEGL; and pEGF.

9. The peptide of claim 2, wherein R¹ is any one selected from H, COCH₃ and a bond to another amino acid.

10. The peptide of claim 2, wherein N(R⁴)₂ is NH₂, NH(CH₃) or NH(CH₂CH₃).

11. The peptide of claim 2, wherein N(R^{4′})₂ is NH₂, N(CH₃)₂, NH(CH₃) or NH(CH₂CH₃).

12. The peptide of claim 2, comprising formula (I) and formula (II).

13. The peptide of claim 12, wherein the peptide is represented by any one of formulae (IIIa) to (IIIc) and (IVa).

14. The peptide of claim 12, wherein the peptide is represented by formula (IIIa).

15. The peptide of claim 1, comprising formula (I) or formula (II).

16. The peptide of claim 15, wherein the peptide comprises 3 or 4 amino acids, such as a peptide represented by any one of formulae (I), (II), and (Va) to (Vd).

17. The peptide of claim 15, wherein the peptide is of formula (I).

18. The peptide of claim 1, wherein the peptide is of any one sequence selected from the group consisting of SEQ ID NO. 2 to 18.

19. The peptide of claim 15, wherein the peptide is of formula (II).

20. The peptide of claim 1, wherein the peptide is of any one sequence selected from the group consisting of SEQ ID NO. 19 to 33.

21. The peptide of claim 15, wherein the peptide comprises 4 to 7 amino acids.

22. The peptide of claim 21, wherein the peptide is represented by any one of formulae (Va), (Vc), (Vd), (VIa), (VId) to (VIf), (VIIa), (VIIe) to (VIIh), (VIIIa), and (VIIIf) to (VIIIj).

23. in A method comprising administering an effective amount of the peptide of claim 1 for regulating leukocyte migration.

24. The method of claim 23, wherein the peptide is administered to inhibit leukocyte migration and a level of inhibition of migration is such that migration is reduced by at least about 30%.

25. The peptide of claim 24, wherein the migration of leukocytes is trans-endothelial.

26. A method comprising administering an effective amount of the peptide of claim 1 to a subject having inflammation and/or musculoskeletal loss and/or damage.

27. The method of claim 26, wherein the inflammation is a symptom of an immune-mediated inflammatory disease, allergic disease or neutrophil mediated disease.

28. The method of claim 27, wherein the immune-mediated inflammatory disease is selected from the group consisting of: dry eye disease, anterior and posterior uveitis (ocular disease), atopic keratoconjunctivitis, vernal keratoconjunctivitis, seasonal and perennial allergic conjunctivitis, eye inflammation post surgery and laser treatment, inflammation caused by gene therapy vectors and other biologics, viral inflammation, systemic lupus erythematosus, virally induced T cell driven cytokine storm such as septicaemia, acute respiratory distress syndrome (ARDS), chronic obstructive pulmonary disease (COPD), lung fibrosis such as idiopathic pulmonary fibrosis (IPF), rheumatoid arthritis, psoriatic arthritis, JIA, Crohn's disease, inflammatory bowel disease (IBD), psoriasis, systemic lupus erythematosus, type I diabetes mellitus, multiple sclerosis, ulcerative colitis, systemic sclerosis, sinusitis, graft versus host disease, asthma, allergies, Sjogren's syndrome, photodermatitis, ankylosing spondylitis, lymphoid interstitial pneumonitis, Peyronie's disease, Behcet's disease, inflammatory and fibrotic liver disease(s) including steatohepatitis, autoimmune hepatitis and cirrhosis, sarcoidosis, giant cell arteritis, uveitis (ocular disease), septicaemia and ischaemia/reperfusion injury.

29. The method of claim 28, wherein the dry eye disease is selected from any one of the group consisting of hypolacrimation, tear deficiency, xerophthalmia, Sjogren's syndrome dry eye, non-Sjogren's syndrome dry eye, keroconjunctivitis sicca, aqueous tear-deficiency dry eye (ADDE), evaporative dry eye (EDE), environmental dry eye, Stevens-Johnson syndrome, ocular pemphigoid blepharitis marginal, eyelid-closure failure, sensory nerve paralysis, allergic conjunctivitis-associated dry eye, post-viral conjunctivitis dry eye, post-cataract surgery dry eye, VDT operation-associated dry eye, and contact lens wearing-associated dry eye.

30. The method of claim 29, wherein the musculoskeletal loss and/or damage is associated with osteoporosis and/or bone injury.

31. The method of claim 30, wherein the osteoporosis results from any one or a combination of the group consisting of aging; prolonged bed rest; space travel; autoimmune disorders including rheumatoid arthritis, psoriatic arthritis, osteoarthritis and JIA; genetic disorders including cystic fibrosis, Ehlers-Danlos, glycogen storage diseases, Gaucher's disease, homocystinuria, hypophosphatasia, idiopathic hypercalciuria, Marfan syndrome, Menkes steely hair syndrome, osteogenesis imperfect, porphyria and Riley-Day syndrome; hypogonadal states including androgen insensitivity, anorexia nervosa, athletic amenorrhea, hyperprolactinemia, panhypopituitarism, premature ovarian failure and Turner's and Klinefelter's syndrome; endocrine disorders including acromegaly, adrenal insufficiency, Cushing's Syndrome, Diabetes Mellitus (Type 1), hyperparathyroidism and thyrotoxicosis; gastrointestinal diseases including gastrectomy, inflammatory bowel disease, malabsorption, celiac disease and primary biliary cirrhosis; hematologic disorders including haemophilia, leukemias and lymphomas, multiple myeloma, sickle cell disease, systemic mastocytosis and thalassemia; rheumatic and auto-immune diseases including ankylosing spondylitis, lupus and rheumatoid arthritis; alcoholism; amyloidosis; chronic metabolic acidosis; congestive heart failure; depression; emphysema; end stage renal disease; epilepsy; idiopathic scoliosis; immobilisation; multiple sclerosis; muscular dystrophy; post-transplant bone disease; and sarcoidosis.

32. The method of claim 31 wherein the bone injury is associated with sports injuries or any one or a combination of neurological disorders including stroke, multiple sclerosis, cerebral palsy, Parkinson's disease, spinal cord injury, neuropathy, sciatica and dementia; delirium; dizziness; vertigo; and dehydration.

33. The method of claim 32, wherein the musculoskeletal loss and/or damage is bone fracture.

34. A method of reducing bone loss and/or stimulating bone production, the method comprising administering an effective amount of the peptide defined in claim 1 ex vivo directly to bone cells and/or their precursors.

35. A pharmaceutical composition comprising a therapeutically effective amount of the peptide defined in claim 1 and a pharmaceutically acceptable excipient.
